(19) United States
(12) Patent Application Publication
(10) Pub. No.: US 2025/0163485 A1
(43) Pub. Date: May 22, 2025

(54) ENZYMATIC SYNTHESIS OF POLYNUCLEOTIDE

(71) Applicant: CHENG-YAO CHEN, Hsinchu City (TW)

(72) Inventors: CHENG-YAO CHEN, Hsinchu City (TW); YI-WEN CHENG, Hsinchu City (TW); TSU-YING WU, Hsinchu City (TW); WEN-TING CHEN, Hsinchu City (TW)

(73) Assignee: CHENG-YAO CHEN, Hsinchu City (TW)

(21) Appl. No.: 18/833,402

(22) PCT Filed: Jan. 18, 2023
(86) PCT No.: PCT/US2023/060847

§ 371 Date: Jul. 26, 2024

Related U.S. Application Data
(60) Provisional application No. 63/304,282, filed on Jan. 28, 2022.

(51) Int. Cl. C12P 19/34; C12N 9/22; C12N 15/11

(52) U.S. Cl. CPC C12P 19/34; C12N 9/22; C12N 15/11; C12N 2310/20

(57) ABSTRACT

Provided is a method and a kit for synthesizing and retrieving a polynucleotide enzymatically, including an initiator containing having a free 3′-hydroxyl group; a polymerase for incorporating nucleotide monomers to the initiator to form a nucleic acid strand from the free 3′-hydroxyl group, wherein the nucleic acid strand includes a guiding nucleotide to be recognized by an endonuclease; and the endonuclease cleaving the nucleic acid strand to release a predetermine polynucleotide and newly form a free 3′-hydroxyl group at 3′ end of the remaining nucleic acid strand which serve as a reusable initiator. Also provided is a kit for synthesizing and retrieving a polynucleotide using the method.

### CROSS REFERENCE

This application claims priority to, and the benefit of, U.S. Provisional Application No. U.S. 63/304,282, filed on Jan. 28, 2022, the entire content thereof is incorporated herein by reference.

### SEQUENCE LISTING

Pursuant to 37 CFR § 1.831-835, the instant application contains a computer readable Sequence Listing which has been submitted electronically in XML format and is hereby incorporated by reference in its entirety. Said XML format file, created on Jan. 18, 2023, is named Sequence Listing.xml and is 20.8 kb in size.

### TECHNICAL FIELD

The present disclosure relates to a method for enzymatic synthesis of nucleic acids or polynucleotides under various conditions, and a kit for implementation of such method.

### BACKGROUND

De novo enzymatic nucleic acid synthesis approaches are rapidly developed to meet the surging demands of making user-defined sequence and length of nucleic acids or polynucleotides for various emerging applications, such as synthetic biology, next-generation sequencing, nucleic acid-based therapeutics, diagnostics, vaccines, DNA data storage, and so forth.

Currently, the enzymatic nucleic acid synthesis approach relies on a template-independent polymerase, such as terminal deoxynucleotidyl transferase (TdT) to repeatedly add nucleotides to an initiator. The initiator, which is normally composed of a single-stranded nucleic acid, or polynucleotide, serves as a starting point for synthesizing new nucleic acids or polynucleotide. Normally, the synthesis initiator needs to be immobilized on a solid support. Hence, the enzymatic nucleic acid synthesis can continue to elongate the nucleic acid strand or polynucleotide chain from the initiator until the desired length and sequence are obtained.

When devising a practical implementation of such enzymatic synthesis of nucleic acids, one of the major challenges is to efficiently retrieve the newly synthesized oligonucleotide/polynucleotide product from the immobilized initiator on the solid support. For instance, U.S. Pat. No. 10,683,536 B2 describes an enzymatic method for synthesizing polynucleotides, which requires a specific cleavage agent, such as an alkaline solution, a metal ion, and a type II restriction endonuclease, to decouple the newly synthesized nucleic acid or polynucleotide from the nucleic acid initiator. However, the release of nascent nucleic acid or polynucleotide utilizing the alkaline solution requires a unique, chemical linker between the newly synthesized nucleic acid and the initiator for the site-specific cleavage reaction, which is inconvenient for routine implementation. In another aspect, the disclosure regarding the cleavage of nascent nucleic acid or polynucleotide from an initiator using the type II restriction endonuclease still lack examples to prove the feasibility. Also, it is known in the art that the type II restriction endonuclease requires a sequence-specific recognition site, such as a palindromic sequence typically with 4 to 8 bases, for DNA strand cleavage, which limits its broad adaptations in de novo enzymatic nucleic acid synthesis.

Furthermore, US Patent Application Publication No. 2021/0254114 A1 describes a method for cleaving a polynucleotide product of template-free enzymatic nucleic acid synthesis, in which an initiator is devised to harbor a site-specific 3′-penultimate deoxyinosine (dI). The position of dI in the initiator can be recognized by endonuclease V (EndoV) and served as a guide for EndoV to precisely cleave the newly synthesized DNA strand from the initiator. According to this disclosure, the DNA-strand cleavage activity is solely achieved by E. coli endonuclease V (EcoEndoV) and the overall performance is comparable to the two-enzyme-based USER method, which adopts the deoxyuridine excision by E. coli uracil-DNA glycosylase in combination of the DNA-strand cleavage by Endonuclease VIII to release the DNA strand from the initiator. However, both EcoEndoV and USER approaches are constrained to the deoxyinosine and deoxyuridine, respectively, for the site-specific recognition in the initiator. Furthermore, both EcoEndoV and USER's enzymatic reactions have to perform at moderate reaction temperatures (<50° C.) and are inapplicable for the nucleic acid-cleavage reactions at higher temperatures (≥50° C.). The cleavage capability of these two enzyme systems fail to meet the users' demands in adopting the de novo polynucleotide synthesis for diverse conditions and applications.

Thus, there is an urgent need for more efficient, diverse, cost-effective, and thermotolerant approaches to retrieve the newly synthesized nucleic acid strand, or polynucleotide chain, and simultaneously generate a new initiator with a free hydroxyl group in one step, which allows for the next round of enzymatic nucleic acid synthesis.

### SUMMARY

Provided herein discloses a method and a kit of preparing synthetic polynucleotides for multiple applications, for example, for the preparation of custom polynucleotides, DNA or RNA probes.

Provided herein discloses a site-specific retrieving method and kit to obtain predetermined sequence and length of polynucleotides according to user's intention. By adopting the approach disclosed herein, the user may configure the sequence and length of the predetermined polynucleotides as well as the position of a guiding nucleotide in the newly synthesized nucleic acids and directly retrieve the desired predetermined polynucleotides by a site-specific polynucleotide cleavage using a single enzyme. Therefore, the method and a kit disclosed herein enable the user to obtain the predetermined sequence and length of polynucleotide precisely and efficiently.

The method provided herein is a method of synthesizing a polynucleotide enzymatically, the method comprises: providing an initiator comprising a 3′-terminal nucleotide having a free 3′-hydroxyl group: incorporating nucleotide monomers to the initiator by a polymerase to elongate a nucleic acid strand from the free 3′-hydroxyl group, wherein the nucleotide monomers comprise a guiding nucleotide to be recognized by an endonuclease, such that the guiding nucleotide is incorporated at a specific position of the newly synthesized nucleic acid strand; and subjecting the endonuclease to cleave the nucleic acid strand according to a position of the guiding nucleotide to release a predetermined sequence and length of polynucleotide and leaves a remaining nucleic acid strand or the initiator having a free 3′-hydroxy group, wherein the endonuclease specifically recognizes the position of guiding nucleotide in the newly synthesized nucleic acid strand and cleaves at the second phosphodiester bond 3′ to the guiding nucleotide, the first phosphodiester bond 5′ to the guiding nucleotide, the second phosphodiester bond 5′ to the guiding nucleotide, or the third phosphodiester bond 5′ to the guiding nucleotide, so that the predetermined sequence and length of desired polynucleotide is obtained and the remaining nucleic acid strand having a free 3′-hydroxyl group, which can be served as a new initiator for another round of polynucleotide synthesis.

In some embodiments, the method provided herein is used in a template-independent or a template-dependent (i.e., templated-directed) polynucleotide synthesis. In a template-independent polynucleotide synthesis, the initiator is a single-stranded nucleic acid having a free 3′ hydroxyl group for elongating a nascent nucleic acid strand. In a template-dependent polynucleotide synthesis, the initiator is a single-stranded primer annealed with a complimentary template to form a primer-template duplex for directing the nucleic acid synthesis by polymerase. The polymerase elongates the primer according to the sequence information of the template.

In some embodiments, the guiding nucleotide is a natural, unnatural, or modified nucleotide, wherein the nucleobase of the nucleotide may be, for example, an uracil, a xanthine, or a hypoxanthine.

In some embodiments, the guiding nucleotide is a natural, unnatural, or modified nucleotide, such as nucleotide containing an uracil or inosine.

In some embodiments, the method provided herein comprises the use of a template-independent or a template-dependent polymerase, wherein the template independent polymerase may be, for example, a B-family DNA polymerase.

Also provided herein is a method of retrieving a polynucleotide enzymatically, the method comprising: providing a synthesized polynucleotide having a guiding nucleotide to be recognized specifically by an endonuclease; and subjecting the endonuclease to cleave the synthesized polynucleotide to release a predetermined sequence and length of polynucleotide, wherein the endonuclease recognizes position of the guiding nucleotide in the synthesized polynucleotide and cleaves at the second phosphodiester bond 3′ to the guiding nucleotide, the first phosphodiester bond 5′ to the guiding nucleotide, the second phosphodiester bond 5′ to the guiding nucleotide, or the third phosphodiester bond 5′ to the guiding nucleotide, so that the predetermined sequence and length of polynucleotide is obtained.

In some embodiments, the endonuclease derives from Thermococcus barophilus (Tba), Pyrococcus furiosus (Pfu), Methanosarcina acetivorans (Mac). Bacillus pumilus (Bpu), Pyrococcus abyssi (Pab), Thermococcus kodakarensis (Tko), Thermococcus gammatolerans (Tga), or Bacillus subtilis (Bsu).

In some embodiments, the method provided herein comprises use of a hyperthermophilic or mesophilic endonuclease capable of specifically recognizing an incorporated guiding nucleotide in a newly synthesized nucleic acid strand or polynucleotide chain, therefore the endonuclease and cleave the nucleic acid strand or polynucleotide chain according to the position of guiding nucleotide in the nucleic acid strand or polynucleotide chain, wherein the second phosphodiester bond 3′ to the guiding nucleotide, the first phosphodiester bond 5′ to the guiding nucleotide, the second phosphodiester bond 5′ to the guiding nucleotide, or the third phosphodiester bond 5′ to the guiding nucleotide can be specifically cleaved by the corresponding endonuclease, respectively.

In some embodiments, the hyperthermophilic endonuclease derives from the group consisting of Thermococcus barophilus endonuclease V (Tba Endo V), Pyrococcus furiosus endonuclease V (Pfu Endo V), Thermococcus kodakarensis endonuclease V (Tko Endo V), Pyrococcus furiosus endonuclease Q (Pfu Endo Q), Methanosarcina acetivorans endonuclease Q (Mac Endo Q), Pyrococcus abyssi NucS endonuclease (Pab NucS), Thermococcus kodakarensis EndoMS endonuclease (Tko EndoMS), Thermococcus gammatolerans NucS endonuclease (Tga NucS) and the enzyme variants or mutants thereof.

In some embodiments, the method provided herein comprises use of a mesophilic endonuclease from the group such as Bacillus subtilis endonuclease V (Bsu Endo V; SEQ ID NO: 11), Bacillus pumilus endonuclease Q (Bpu Endo Q: SEQ ID NO: 12), and the enzyme variants thereof in the newly synthesized nucleic acid strand or polynucleotide chain.

In some embodiments, the endonuclease derives from the group consisting of Bacillus subtilis endonuclease V (Bsu Endo V), Escherichia coli endonuclease V (Eco Endo V: SEQ ID NO: 7), Pyrococcus furiosus endonuclease V (Pfu Endo V; SEQ ID NO: 8), Thermococcus barophilus endonuclease V (Tba Endo V; SEQ ID NO: 10), Thermococcus kodakarensis endonuclease V (Tko Endo V: SEQ ID NO: 16) or the enzyme variants thereof and specifically cleaves at the second phosphodiester bond 3″ to the guiding nucleotide.

In some embodiments, the endonuclease is selected from the group consisting of Pyrococcus furiosus endonuclease Q (Pfu Endo Q: SEQ ID NO: 9), Methanosarcina acetivorans endonuclease Q (Mac Endo Q: SEQ ID NO: 14), Bacillus pumilus endonuclease Q (Bpu Endo Q: SEQ ID NO: 12) and the enzyme variants thereof and specifically cleaves at the first phosphodiester bond 5′ to the guiding nucleotide in the newly synthesized nucleic acid strand or polynucleotide chain.

In some embodiments, the endonuclease derives from the group consisting of Thermococcus gammatolerans NucS endonuclease (Tga NucS: SEQ ID NO: 15), Pyrococcus abyssi NucS endonuclease (Pab NucS: SEQ ID NO: 13) and the enzyme variants thereof and specifically cleaves at the second phosphodiester bond 5′ to the guiding nucleotide in the newly synthesized nucleic acid strand or polynucleotide chain.

In some embodiments, the endonuclease is selected from the group consisting of Thermococcus kodakarensis EndoMS endonuclease (Tko EndoMS: SEQ ID NO: 17) and the enzyme variants thereof and specifically cleaves at the third phosphodiester bond 5′ to the guiding nucleotide in the newly synthesized nucleic acid strand or polynucleotide chain.

In some embodiments, the endonuclease recognizes a guiding nucleotide in the nascent nucleic acid strand or the synthesized polynucleotide to cleave at specific phosphodiester bond near the position of guiding nucleotide. In some embodiments, the method provided herein may be implemented at higher temperatures compared to those conventionally used. The temperature range for implementation can be, for example, from 10° C. to 100° C., from 50° C. to 90° C., or from 70° C. to 90° C. In some embodiments, the nucleic acid initiator, or the synthesized/synthetic polynucleotide in a free form or duplex formation of the method provided herein is attached to a solid support and immobilized via its 5′ end. The solid support can be, for example, a particle, a resin, a bead, a slide, a chip, an array, a membrane, a matrix, a flow cell, a well, a chamber, a microfluidic chamber, a channel, a microfluidic channel, a gel, a synthetic polymer, or any surface that can be attached with a synthetic nucleic acid strand or polynucleotide.

The present disclosure further provides a kit for synthesizing a polynucleotide enzymatically. In some embodiments, the sequence and length of polynucleotide is predetermined or designed. The kit for synthesizing predetermined sequence and length of oligonucleotides of the present disclosure may comprise: a nucleic acid initiator comprising a 3′-terminal nucleotide having a free 3′-hydroxyl group: a polymerase for incorporating a plurality of nucleotide monomers to the initiator to elongate a nucleic acid strand from the free 3′-hydroxyl group of the initiator; wherein nucleotide monomers comprises a guiding nucleotide to be specifically recognized by an endonuclease, such that the guiding nucleotide is incorporated in the defined position of nucleic acid strand; and the endonuclease cleaves the nucleic acid strand according to the position of the guiding nucleotide to release a desired sequence and length of polynucleotide and leave a free 3′-hydroxyl group at the 3′ end of a remaining nucleic acid strand, wherein the endonuclease specifically recognizes the guiding nucleotide and cleaves at the second phosphodiester bond 3′ to the guiding nucleotide, the first phosphodiester bond 5′ to the guiding nucleotide, the second phosphodiester bond 5′ to the guiding nucleotide, or the third phosphodiester bond 5′ to the guiding nucleotide, respectively. Hence the desired sequence and length of polynucleotide is obtained and the remaining nucleic acid strand having the new free 3″-hydroxyl group can be served as a new initiator for another round of polynucleotide synthesis.

The present disclosure further provides a kit for retrieving a predetermined sequence and length of polynucleotide enzymatically. In some embodiments, the targeting polynucleotide to be cleaved is any synthetic polynucleotide prepared in advance. In some embodiment the kit comprises the synthetic polynucleotide, which has a site-specific guiding nucleotide; and an endonuclease for cleaving the synthetic polynucleotide to release a predetermined polynucleotide, wherein the endonuclease recognizes the position of the guiding nucleotide in the polynucleotide and cleaves at the second phosphodiester bond 3′ to the guiding nucleotide, the first phosphodiester bond 5′ to the guiding nucleotide, the second phosphodiester bond 5′ to the guiding nucleotide, or the third phosphodiester bond 5′ to the guiding nucleotide, respectively, so that the desired predetermined polynucleotide is obtained.

In some embodiments, the guiding nucleotide containing an uracil or inosine in the nucleic acid strand or a polynucleotide chain is either pre-existing or newly synthesized chemically or enzymatically.

### BRIEF DESCRIPTION OF THE DRAWINGS

FIG. 1 is a schematic diagram in accordance with at least one embodiment of the present disclosure, illustrating a de novo template-independent single-stranded DNA (ssDNA) synthesis from an initiator having a free 3′-hydroxyl group at 3′ end thereof, the cleavage of the newly synthesized DNA strand to obtain a desired sequence and length of polynucleotide, and the regeneration of a new free 3′-hydroxyl group of the remaining nucleic acid strand after cleavage of the synthesized DNA strand to obtain a predetermined polynucleotide and to serve as a new reusable initiator for the next round of nucleic acid synthesis. The 5′ end of the initiator is immobilized to the solid support (SS) prior to the nucleic acid synthesis reaction. X represents the guiding nucleotide and is used as the origin to number nucleotide position in the nucleic acid strand. N stands for an incorporated nucleotide monomer, in which the subscript numbers in ascending order (e.g., N₁, N₂, N₃, N₄, and N₅) to the 3′ end or downstream of the strand and the subscript numbers in descending order (e.g., N₋₁, N₋₂, and N₋₃) to the 5′ end or upstream of the strand. E₁ to E₄ stand for different endonucleases. The scheme depicts an enzymatic nucleic acid synthetic comprising an enzymatic cleavage that may be conducted by E₁, E₂, E₃, or E₄ at the phosphodiester linkage between N₂ and N₁, X and N₋₁, N₋₁ and N₋₂, or N₋₂ and N₋₃, respectively, wherein the 5′ end of the initiator is attached to the solid support (SS).

FIGS. 2A and 2B are schematic diagrams in accordance with at least one embodiment of the present disclosure, illustrating template-directed DNA synthesis from an initiator (i.e., primer) having a free 3′-hydroxyl group at 3′ end thereof,: the cleavage of the nascent DNA strand to obtain a desired sequence and length of polynucleotide and the regeneration of a free 3′-hydroxyl group at 3′ end of the remaining nucleic acid strand to form a new reusable initiator for the next round of nucleic acid synthesis. The 5′ end of the initiator is immobilized to the solid support (SS) prior to the nucleic acid synthesis reaction. Accordingly, the template DNA is attached to the solid support via the 3′-end (FIG. 2A) or partially hybridized to the initiator by annealing method or other known hybridization techniques (FIG. 2B). X represents the guiding nucleotide and is used as the origin to number the nucleic acid strand. N stands for an incorporated nucleotide monomer, in which the subscript numbers in ascending order (e.g., N₁, N₂, N₃, and N₄) to the 3′ end or downstream of the strand and the subscript numbers in descending order (e.g., N₋₁, N₋₂, and N₋₃) to the 5′ end or upstream of the strand. E₁ to E₄ stand for different endonucleases. The scheme depicts an enzymatic synthetic scheme comprising an enzymatic cleavage that may be conducted by E₁, E₂, E₃, and E₄ at the phosphodiester linkage between N₂ and N₁, X and N₋₁, N₋₁ and N₋₂, or N₋₂ and N₋₃, respectively.

FIG. 3 is the fluorescent image of urea-polyacrylamide gel, showing the feasibility of the template-independent nucleic acid synthesis in accordance with at least one embodiment of the present disclosure. Lane S refers to the initiator DNA only (Biotin-FAM-45-mer ssDNA); lane 1 refers to the initiator DNA elongated with 3′-O-(azidomethyl)-2′-deoxyuridine (U) by the polymerase; lane 2 refers to the initiator DNA elongated with 3′-O-(azidomethyl)-2′-deoxyuridine (U) and 3′-O-(azidomethyl)-2′-deoxyguanosine (G) consecutively, after each nucleotide-incorporation and 3′-deprotection steps; and lane 3 refers to the initiator DNA elongated with a 3′-O-(azidomethyl)-2′-deoxyuridine followed with dNTPs (dATP, dCTP, dGTP, and dTTP mixture) after the 3′-deprotection step.

FIGS. 4A and 4B are the fluorescent images of urea-polyacrylamide gels showing the results of Example 1 and 2, respectively, which illustrates the site-specific cleavage of the free form (FIGS. 4A (1) and 4B (1), or immobilized form (FIGS. 4A (2) and 4B (2)) of the single-stranded DNA (ssDNA), and the free form (FIGS. 4A (3) and 4B (3)) or immobilized form (FIGS. 4A (4) and 4B (4)) of the double-stranded DNA (dsDNA) by exemplary enzymes that preferentially or specifically recognize deoxyinosine (I, FIG. 4A) or deoxyuridine (U, FIG. 4B), respectively, in the DNA under 37° C. or 70° C. S refers to the substrate DNA only: Eco Endo V refers to the in-house prepared E. coli endonuclease V enzyme, which can be referenced to US 2021/0254114A1; Pfu EndoV refers to Pyrococcus furiosus endonuclease V: CI refers to the commercial endonuclease V from New England Biolabs (NEB Eco EdoV) (Cat. #M0305S, Ipswich, MA): C2 refers to the in-house developed enzyme mixture containing human alkyladenine DNA glycosylase (hAAG) and Endo VIII; and C3 refers to an uracil-specific excision reagent from New England Biolabs (NEB USER) (Cat. #M5505S, Ipswich, MA).

FIGS. 5A and 5B are the fluorescent images of urea-polyacrylamide gels showing the results of Example 3 and 4, respectively, which illustrate the site-specific cleavage of the free form (FIGS. 5A (1) and 5B (1), or immobilized form (FIGS. 5A (2) and 5B (2)) of the single-stranded DNA (ssDNA), and the free form (FIGS. 5A (3) and 5B (3)) or immobilized form (FIGS. 5A (4) and 5B (4)) of the double-stranded DNA (dsDNA) by the exemplary enzymes that preferentially or specifically recognize deoxyinosine (I, FIG. 5A) or deoxyuridine (U, FIG. 5B), respectively, in the DNA under 37° C. or 70° C. Pfu EndoQ refers to Pyrococcus furiosus endonuclease Q: CI refers to the commercial endonuclease V from New England Biolabs (NEB Eco EdoV) (Cat. #M0305S, Ipswich, MA): C2 refers to the in-house developed enzyme mixture containing human alkyladenine DNA glycosylase (hAAG) and Endo VIII; and C3 refers to an uracil-specific excision reagent from New England Biolabs (NEB USER) (Cat. #M5505S, Ipswich, MA).

FIGS. 6A and 6B are fluorescent images of urea-polyacrylamide gels showing the results of Example 5 and 6, respectively, which illustrate the site-specific cleavage of the free form (FIGS. 6A (1) and 6B (1), or immobilized form (FIGS. 6A (2) and 6B (2)) of the single-stranded DNA (ssDNA), and the free form (FIGS. 6A (3) and 6B (3)) or immobilized form (FIGS. 6A (4) and 6B (4)) of the double-stranded DNA (dsDNA) by the exemplary enzymes that preferentially or specifically recognize deoxyinosine (I, FIG. 6A) or deoxyuridine (U, FIG. 6B), respectively, in the DNA under 37° C. or 70° C. S refers to the substrate DNA only: Eco Endo V refers to the in-house prepared E. coli endonuclease V enzyme, which can be referenced to US 2021/0254114A1: Tba Endo V refers to Thermococcus barophilus endonuclease V: CI refers to the commercial endonuclease V from New England Biolabs (NEB Eco EdoV) (Cat. #M0305S, Ipswich, MA): C2 refers to the in-house developed enzyme mixture containing human alkyladenine DNA glycosylase (hAAG) and Endo VIII; and C3 refers to an uracil-specific excision reagent from New England Biolabs (NEB USER) (Cat. #M5505S, Ipswich, MA).

FIG. 7 is the fluorescent image of urea-polyacrylamide gel showing the results of Example 7, which illustrate the site-specific cleavage of the free form (FIGS. 7 (1)) of the single-stranded DNA (ssDNA) and free form (FIGS. 7A (2)) of the double-stranded DNA (dsDNA) by the exemplary enzymes that preferentially or specifically recognize deoxyuridine (U) in the DNA at 37° C. S refers to the substrate DNA only: Eco Endo V refers to the in-house prepared E. coli endonuclease V enzyme, which can be referenced to US 2021/0254114A1: Bsu Endo V refers to Bacillus subtilis endonuclease V: C1 refers to the commercial endonuclease V from New England Biolabs (NEB Eco EdoV) (Cat. #M0305S, Ipswich, MA); and C3 refers to an uracil-specific excision reagent from New England Biolabs (NEB USER) (Cat. #M5505S, Ipswich, MA).

FIGS. 8A and 8B are fluorescent images of urea-polyacrylamide gels showing the results of Example 8, which illustrate the site-specific cleavage of elongated DNA strand product from the free form (FIGS. 8A (1), 8A (3), 8B (1), and 8B (3)), or the immobilized form of the (FIGS. 8A (2), 8A (4), 8B (2), and 8B (4)) single-stranded DNA (ssDNA) by the exemplary enzymes that preferentially or specifically recognize deoxyinosine (I) (FIGS. 8A (1), 8A (2), 8B (1), and 8B (2)) and deoxyuridine (U) (FIGS. 8A (3), 8A (4), 8B (3), and 8B (4)), respectively, in the DNA at 37° C., 55° C., and/or 60° C. S refers to the substrate DNA only: Eco Endo V refers to the in-house prepared E. coli endonuclease V enzyme, which can be referenced to US 2021/0254114A1; Pfu Endo V refers to Pyrococcus furiosus endonuclease V: Tba refers to Thermococcus barophilus endonuclease V: Pfu Endo Q refers to Pyrococcus furiosus endonuclease Q; and Bpu Endo Q refers to Bacillus pumilus endonuclease Q.

### DETAILED DESCRIPTION

The following embodiments are provided to illustrate the present disclosure in detail. A person having ordinary skill in the art can easily understand the advantages and effects of the present disclosure after reading the disclosure of this specification and can implement or apply in other different embodiments. Therefore, it is possible to modify and/or alter the following embodiments for carrying out this disclosure without contravening its scope for different aspects and applications, and any element or method within the scope of the present disclosure disclosed herein can combine with any other elements or methods disclosed in any embodiments of the present disclosure.

As used herein, the singular forms “a,” “an,” and “the” are intended to include the plural forms, unless the context clearly indicates otherwise, and the term “or” is used interchangeably with the term “and/or” unless the context clearly indicates otherwise.

As used herein, the terms “including,” “comprising,” “containing,” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

The numeral ranges used herein are inclusive and combinable, any numeral value that falls within the numeral scope herein could be taken as a maximum or minimum value to derive the sub-ranges therefrom. For example, the numeral range “from 10° C. to 100° C.” comprises any sub-ranges between the minimum value of 10° C. to the maximum value of 100° C., such as the sub-ranges from 10° C. to 50° C., from 60° C. to 100° C., from 70° C. to 90° C. and so on. In addition, a plurality of numeral values used herein can be optionally selected as maximum and minimum values to derive numerical ranges. For instance, the numerical ranges of 37° C. to 55° C., 37° C. to 70° C., and 55° C. to 70° C. can be derived from the numeral values of 37° C., 55° C., and 70° C.

As used herein, the term “about” generally referring to the numerical value meant to encompass variations of ±20%, ±10%, ±5%, ±1%, ±0.5%, or ±0.1% from a given value or range. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations. Alternatively, the term “about” means within an acceptable standard error of the mean when considered by one of ordinary skill in the art. Unless otherwise expressly specified, all of the numerical ranges, amounts, values and percentages such as those for quantities of materials, durations of time periods, temperatures, operating conditions, ratios of amounts, and the likes disclosed herein should be understood as modified in all instances by the term “about.”

The terms “nucleic acid,” “nucleic acid sequence,” and “nucleic acid fragment” as used herein refer to a guiding nucleotide or ribonucleotide sequence in a single-stranded or double-stranded form, of which the sources are not limited herein, and generally, include naturally occurring nucleotides or artificial chemical mimics. The term “nucleic acid” as used herein is interchangeable with the terms including natural or unnatural “oligonucleotide”, “polynucleotide”, “gene”, “cDNA”, “RNA”, and “mRNA”.

The nucleic acid as used herein also includes nucleic acid analogue. The term nucleic acid analogue is known to describe compounds or artificial nucleic acids which are functionally or structurally equivalent to naturally existing RNA and DNA. A nucleic acid analogue may have one or more parts of a nucleotide (the phosphate backbone, pentose sugar, and nucleobase) being modified. These modifications on the nucleotide change the structure and geometry of the nucleic acid and its interactions with nucleic acid polymerases. The nucleic acid analogue also encompasses the emerging category of artificial nucleic acids, such as xeno nucleic acids (XNAs), which is designed to have new-to-nature forms of sugar backbone.

Examples of nucleic acid analogues include but are not limited to: the universal bases, such as inosine, 3-nitropyrrole, and 5-nitroindole, which can form a base-pair with all four canonical bases; the phosphate-sugar backbone analogues, such as peptide-nucleic acids (PNA), which affect the backbone properties of the nucleic acid; chemical linker or fluorophore-attached analogues, such as amine-reactive aminoallyl nucleotide, thiol-containing nucleotides, biotin-linked nucleotides, rhodamine-linked nucleotides, and cyanine-linked nucleotides; the fluorescent base analogues, such as 2-aminopurine (2-AP), 3-methylisoxanthopterin (3-MI), 6-methylisoxanthopterin (6-MI), 4-amino-6-methylisoxanthopterin (6-MAP), and 4-dimethylaminopyridine (DMAP); the nucleic acid probes for various genetic applications, such as the oligonucleotide-conjugated with a fluorescent reporter dye (ALEXA, FAM, TET, TAMRA, CY3, CY5, VIC, JOE, HEX, NED, PET, ROX, Texas Red and others) and/or a fluorescent quenchers (BHQs); the molecular beacons (MBs), which are single-stranded nucleic acid probes containing a stem-loop structure and a dual fluorophore-and-quencher label; and the nucleic acid aptamers.

The term “oligonucleotide” used herein is generic to any type of polynucleotide including polyribonucleotides, polydeoxyribonucleotides nucleotides, or other polynucleotides that consist of an N-glycoside with a purine or pyrimidine base. The terms “oligonucleotide” and “polynucleotide” used herein are not intended to be distinct in length, where these two terms refer only to the molecule structure, and therefore are used interchangeably herein. An oligonucleotide or polynucleotide can further comprise natural, damaged, or modified nucleotides. Nucleic acid bases contained in an oligonucleotide or polynucleotide may be, for example, adenine, thymine, cytosine, guanine, uracil, xanthine, hypoxanthine, isocytosine, isoguanine, 5-fluorouracil, 5-hydroxymethyluracil, 5-formylcytosine, 5-carboxylcytosine, 3-methyladenine, 3-methylguanine, 7-methyladenine, 7-methylguanine, N6-methyladenine, 8-oxo-7,8-dihydroguanine, 5-hydroxylcytosine, 5-hydroxyluracil, dihydroxyuracil, ethenocytosine, ethenoadenine, thymine glycol, cytosine glycol, 2,6-diamino-4-hydroxy-5-N-methylformamidopyrimidine, a formamidopyrimidine derivative of adenine, a formamidopyrimidine derivative of guanine, adenine opposite guanine, uracil opposite guanine, uracil opposite adenine, thymine opposite guanine, ethenocytosine opposite guanine, adenine opposite 8-oxo-7,8-dihydroguanine, and 2-hydroxyladenine opposite guanine.

The term “endonuclease activity” used herein refers to an enzymatic activity of breaking the linkage bond at a specific and recognizable nucleic acid site, resulting in a cleaving reaction in a single or double stranded DNA. The endonuclease activity may be provided by naturally occurring enzymes and the modified derivatives thereof. The examples of modified derivatives include enzymatically active mutants/variants, fragments, recombinant proteins derived from the enzymes possessing endonuclease activity. For example, an endonuclease may cleave a single-stranded DNA strand and release the oligonucleotide/polynucleotide with a 5′-monophosphate on the one hand and leaves a free 3′-hydroxyl group on the remaining nucleic acid strand on the other hand.

The terms “abasic”, “apurinic/apyrimidinic”, and D-spacer can be interchangeably used to indicate a site at which the base is not present, but the sugar phosphate backbone remains intact. Therefore, the abasic site endonuclease is also known as apurinic/apyrimidinic site endonuclease.

As used herein, the term “template” refers generically to a polynucleotide, or a polynucleotide mimic, which contains the desired or unknown target nucleotide sequence. In some instances, the terms “target sequence,” “template polynucleotide,” “target nucleic acid,” “target polynucleotide,” “nucleic acid template,” “template sequence,” and variations thereof, are used interchangeably. For example, the term “template” refers to a strand of nucleic acid, on which a complimentary copy is synthesized from nucleotides or nucleotide analogues through the replication of a template-dependent, or template-directed, nucleic acid polymerase. Within a nucleic acid duplex, the template strand is, by the convention definition, depicted and described as the “bottom” strand. Similarly, the non-template strand is often depicted and described as the “top” strand. The “template” strand may also be referred to as the “sense” or “plus” strand and the non-template strand as the “antisense” or “minus” strand.

The term “initiator” used herein refers to a mononucleoside, a mononucleotide, an oligonucleotide, a polynucleotide, or modified analogues thereof, from which a nucleic acid is to be synthesized by a nucleic acid polymerase de novo. The term “initiator” may also refer to an XNA or a peptide nucleic acid (PNA) having a 3′-hydroxyl group. The term “primer” used herein refers to a short single-stranded oligonucleotide, a polynucleotide, or a modified nucleic acid analogue used in combination with template by a nucleic acid polymerase to initiate nucleic acid synthesis.

The nucleotide monomer disclosed herein includes canonical nucleotides and nucleotide analogues. The term “nucleotide analogue” is known to those skilled in the art to describe the chemically modified nucleotides or artificial nucleotides, which are structural mimics of canonical nucleotides. These nucleotide analogues can serve as substrates for nucleic acid polymerases to synthesize nucleic acid. A nucleotide analogue may have one or more altered components of a nucleotide (e.g., the phosphate backbone, pentose sugar, and nucleobase), which changes the structure and configuration of a nucleotide and affects its interactions with other nucleobases and the nucleic acid polymerases. For example, a nucleotide analogue having altered nucleobase may confer alternative base-pairing and base-stacking properties in the DNA or RNA. Furthermore, by way of example, the modification at the base may generate various nucleosides such as inosine, methyl-5-deoxycytidine, deoxyuridine, dimethylamino-5-deoxyuridine, diamino-2,6-purine or bromo-5-deoxyuridine, and any other analogues which permits hybridization. In other exemplary aspects, modifications may take place at the level of sugar moiety (for example, replacement of a deoxyribose by an analogue), and/or at the level of the phosphate group (for example, boronate, alkylphosphonate, or phosphorothioate derivatives). A nucleotide analogue monomer may have a phosphate group selected from a monophosphate, a diphosphate, a triphosphate, a tetraphosphate, a pentaphosphate, and a hexaphosphate.

Other examples of nucleotide analogues also include nucleotides having a removable blocking moiety. Examples of the removable blocking moiety include, but are not limited to, a 3′-O-blocking moiety, a base blocking moiety, and a combination thereof. Examples of the 3′-O-blocking moiety include, but are not limited to, O-azido (O—N₃), O-azidomethyl, O-amino, O-allyl, O-phenoxyacetyl, O-methoxyacetyl, O-acetyl, O-(p-toluene) sulfonate, O-phosphate, O-nitrate, O-[4-methoxy]-tetrahydrothiopyranyl, O-tetrahydrothiopyranyl, O-[5-methyl]-tetrahydrofuranyl, O-[2-methyl, 4-methoxy]-tetrahydropyranyl, O-[5-methyl]-tetrahydropyranyl, and O-tetrahydrothiofuranyl, O-2-nitrobenzyl, O-methyl, and O-acyl. Examples of the base blocking moiety may be a reversible dye-terminator. Examples of the reversible dye-terminator include, but are not limited to, a reversible dye-terminator of Illumina MiSeq, a reversible dye-terminator of Illumina HiSeq, a reversible dye-terminator of Illumina Genome Analyzer IIX, a reversible dye-terminator of Helicos Biosciences Heliscope, and a reversible dye-terminator of LaserGen's Lightning Terminators.

The term “polymerase” used herein is generically a DNA polymerase including naturally-occurring enzymes and modified derivatives thereof. For example, a sequence modification to remove 5′ to 3′ or 3′ to 5′ exonuclease activity can be applied to a polymerase. Mutations or deletions to functional groups or sequences of a polymerase are also involved to improve the performance of the polymerase.

According to the present disclosure, the polymerase may be a template-dependent polymerase or a template-independent polymerase. The polymerase may be selected from the group consisting of a family-A DNA polymerase (e.g., T7 DNA polymerase, Pol I, Pol γ, θ, and v), a family-B DNA polymerase (e.g., Pol II, Pol B, Pol ζ, Pol α, δ, and ε), a family-C DNA polymerase (e.g., Pol III), a family-D DNA polymerase (e.g., PolD), a family-X DNA polymerase (e.g., Pol β, Pol σ, Pol λ, Pol μ, and terminal deoxynucleotidyl transferase), a family-Y DNA polymerase (e.g., Pol t, Pol κ, Pol η, DinB, Pol IV, and Pol V), a reverse transcriptase (e.g., telomerase and hepatitis B virus), and enzymatically active fragments thereof.

Non-limiting examples of widely employed template-dependent polymerases include T7 DNA polymerase of the phage T7 and T3 DNA polymerase of the phage T3, which are DNA-dependent DNA polymerases: T7 RNA polymerase of the phage T7 and T3 RNA polymerase of the phage T3, which are DNA-dependent RNA polymerases: DNA polymerase I or its fragment known as the Klenow fragment of Escherichia coli, which is a DNA-dependent DNA polymerase: Thermophilus aquaticus DNA polymerase, Tth DNA polymerase and Vent DNA polymerase, which are thermostable DNA-dependent DNA polymerases: eukaryotic DNA polymerase B, which is a DNA-dependent DNA polymerase: telomerase, which is a RNA-dependent DNA polymerase; and non-protein catalytic molecules, such as modified RNA (ribozymes: Unrau & Bartel, 1998) and DNA with template-dependent polymerase activity.

Non-limiting examples of the template-independent polymerases include reverse transcriptase, poly A polymerase, DNA polymerase theta (0), DNA polymerase mu (u), DpoIV polymerase, and terminal deoxynucleotidyl transferase. Since polymerases suitable for nucleic acid synthesis, linking nucleotide addition, and nucleic acid synthesis are within the expertise and routine skills of those skilled in the art, further details thereof are omitted herein for the sake of brevity.

In some embodiments, the method provided herein comprises use of a B-family polymerase. Examples of a B-family polymerase include, but are not limited to, E. coli DNA polymerase II (Eco), Pseudomonas aeruginosa DNA polymerase II (Pae), Escherichia phage RB69 DNA polymerase (RB69), Escherichia phage T4 DNA polymerase (T4), Bacillus phage Phi29 DNA polymerase (Phi29), Saccharomyces cerevisiae DNA polymerase delta catalytic subunit (ScePOLD), human DNA polymerase delta catalytic p125 subunit (hPOLD), Sulfolobus solfataricus DNA polymerase (Sso), Pyrobaculum islandicum DNA polymerase (Pis), Thermococcus sp. (strain 9°N-7) DNA polymerase (9°N), Thermococcus kodakarensis DNA polymerase (Kod1), Methanococcus maripaludis DNA polymerase (Mma), Pyrococcus furiosus DNA polymerase (Pfu), Thermococcus gorgonarius DNA polymerase (Tgo), and Thermococcus litoralis DNA polymerase (Vent).

The nucleotide monomer used herein may have a removable blocking moiety. Examples of the removable blocking moiety include, but are not limited to, a 3′-O-blocking moiety, a base blocking moiety, and a combination thereof. The nucleotide monomer having a removable blocking moiety is also referred to as a reversible terminator. Therefore, the nucleotide monomer having the 3′-O-blocking moiety is also referred to as a 3′-blocked reversible terminator or a 3′-O-modified reversible terminator, and the nucleotide monomer having a base blocking moiety is also referred to as a 3′-unblocked reversible terminator or a 3′-OH unblocked reversible terminator. As used herein, the term “reversible terminator” refers to a chemically modified nucleotide monomer. When such a reversible terminator is incorporated into a growing nucleic acid by a polymerase, it blocks the further incorporation of another nucleotide monomer by the polymerase. Such “reversible terminator” base and a nucleic acid can be deprotected by chemical or physical treatment, and following such deprotection, the nucleic acid can be further extended by a polymerase. Examples of the 3′-O-blocking moiety include, but are not limited to, O-azidomethyl, O-amino, O-allyl, O-phenoxyacetyl, O-methoxyacetyl, O-acetyl, O-(p-toluene) sulfonate, O-phosphate, O-nitrate, O-[4-methoxy]-tetrahydrothiopyranyl, O-tetrahydrothiopyranyl, O-[5-methyl]-tetrahydrofuranyl, O-[2-methyl, 4-methoxy]-tetrahydropyranyl, O-[5-methyl]-tetrahydropyranyl, and O-tetrahydrothiofuranyl, O-2-nitrobenzyl, O-methylmethyl, and O-acylacyl. Examples of the 3′-unblocked reversible terminators include, but are not limited to, 7-[(S)-1-(5-methoxy-2-nitrophenyl)-2,2-dimethyl-propyloxy]methyl-7-deaza-dATP, 5-[(S)-1-(5-methoxy-2-nitrophenyl)-2,2-dimethyl-propyloxy]methyl-dCTP, 1-[(5-methoxy-2-nitrophenyl)-2,2-dimethyl-propyloxy]-5-methyl-7-deaza-dGTP, 5-[(S)-1-(5-methoxy-2-nitrophen-yl)-2,2-dimethyl-propyloxy]methyl-dUTP, and 5-[(S)-1-(2-nitrophenyl)-2,2-dimethyl-propyloxy]methyl-dUTP. The base blocking moiety may also be a reversible dye-terminator. Examples of the reversible dye-terminator include, but are not limited to, a reversible dye-terminator of Illumina NovaSeq, a reversible dye-terminator of Illumina NextSeq, a reversible dye-terminator of Illumina MiSeq, a reversible dye-terminator of Illumina HiSeq, a reversible dye-terminator of Illumina Genome Analyzer IIX, a lightning terminator of LaserGen, and a reversible dye-terminator of Helicos Biosciences Heliscope.

In some embodiments, the guiding nucleotide is a natural, unnatural, or modified nucleotide, wherein the nucleobase of the nucleotide may be, for example, adenine, thymine, cytosine, guanine, uracil, xanthine, hypoxanthine, isocytosine, isoguanine, 5-fluorouracil, 5-hydroxymethyluracil, 5-formylcytosine, 5-carboxylcytosine, 3-methyladenine, 3-methylguanine, 7-methyladenine, 7-methylguanine, N6-methyladenine, 8-oxo-7,8-dihydroguanine, 5-hydroxylcytosine, 5-hydroxyluracil, dihydroxyuracil, ethenocytosine, ethenoadenine, thymine glycol, cytosine glycol, 2,6-diamino-4-hydroxy-5-N-methylformami dopyrimidine, a formamidopyrimidine derivative of adenine, a formamidopyrimidine derivative of guanine, adenine opposite guanine, uracil opposite guanine, uracil opposite adenine, thymine opposite guanine, ethenocytosine opposite guanine, adenine opposite 8-oxo-7,8-dihydroguanine, and 2-hydroxyladenine opposite guanine. In at least one embodiment, the guiding nucleotide contains a nucleobase selected from the group consisting of uracil, xanthine, hypoxanthine, cytosine, and guanine. In some embodiments, the guiding nucleotide contains an apurinic/apyrimidinic lesion, wherein the apurinic/apyrimidinic lesion is an abasic site or a dSpacer.

Since the reversible terminators are well-known to and commonly used by those skilled in the art, further details of the same are omitted herein for the sake of brevity. Nevertheless, the applicable 3′-blocked reversible terminators, applicable 3′-unblocked reversible terminators, and applicable conditions for protection and deprotection (i.e., conditions for adding and eliminating the removable blocking moiety) can be found in, for example, Gardner et al. (2012), Nucleic Acids Research, 40 (15): 7404-7415, Litosh et al. (2011), Nucleic Acids Research, 39 (6): e39, and Chen et al. (2013), Genomics Proteomics Bioinformatics, 11:34-40.

According to the present disclosure, an initiator or primer-template duplex may be used as the starting material for nucleic acid synthesis. The initiator/primer-template duplex may have a 5′-end linked to a solid support. The initiator may be directly attached to the solid support or may be attached to the solid support via a linker. Examples of the solid support include, but are not limited to, microarrays, beads (coated or non-coated), columns, optical fibers, wipes, nitrocellulose, nylon, glass, quartz, diazotized membranes (paper or nylon), silicones, polyformaldehyde, cellulose, cellulose acetate, paper, ceramics, metals, metalloids, semiconductive materials, magnetic particles, plastics (e.g., polyethylene, polypropylene, and polystyrene), gel forming materials (e.g., gelatins), lipopolysaccharides, silicates, agarose, polyacrylamides, methyl methracrylate polymers), sol-gels, porous polymers, hydrogels, nanostructured surface nanotubes (e.g., carbon nanotubes), and nanoparticles (e.g., gold nanoparticles or quantum dots).

Specifically, when the disclosed method is used under scenario of enzymatical nucleic acid synthesis, a polymerase may elongate a nucleic acid strand by adding a plurality of nucleotide monomers to the initiator, wherein the nucleotide monomers include a site-specific, recognizable nucleotide monomer comprising a predetermined nucleobase or an apurinic/apyrimidinic lesion, which is defined as a guiding nucleotide. During the enzymatic nucleic acid synthesis, the guiding nucleotide is incorporated in the nucleic acid strand at the designated position, and then a site-specific enzymatic cleavage is performed to retrieve the desired target polynucleotide from the newly synthesized nucleic acid strand. Thus, the sequence and length of the nucleic acid strand and the corresponding cleavage enzyme can be flexibly designed and customized in advance to obtain a desired sequence and length of polynucleotide efficiently.

Depending on user's design, the desired nucleic acid strands or polynucleotide to be retrieved may contain newly synthesized nucleic acid or may be the commercially available ready-to-use synthetic nucleic acids. The disclosed cleavage enzymes preferentially or specifically cleave at the specific position of the designed nucleic acid strand to release the desired sequence and length of polynucleotide.

The present disclosure provides a method of synthesizing a defined sequence and length of polynucleotide enzymatically, wherein the method comprises uses of an initiator having a free 3′-hydroxyl group at the 3′ end, a newly synthesized nucleic acid strand by polymerase, and an enzyme/endonuclease having a polynucleotide-cleavage activity, which cleaves a specific phosphodiester bond in the newly synthesized nucleic acid strand. Specifically, a polymerase incorporates nucleotide monomers containing a guiding nucleotide to the initiator to elongate a nucleic acid strand from the free 3′-hydroxyl group. Accordingly, the guiding nucleotide which is incorporated by polymerase at a specific position of the elongated nucleic acid strand. Thereafter, a selected endonuclease recognizes the guiding nucleotide and cleaves the newly synthesized nucleic acid strand according to the position of the guiding nucleotide to release a the predetermined, or desired, sequence and length of polynucleotide.

To cleave the newly synthesized nucleic acid strand for retrieving the predetermined, or desired, sequence and length of polynucleotide, the selected endonuclease specifically recognizes the position of the guiding nucleotide in the nascent nucleic acid strand and preferably cleaves the second phosphodiester bond 3′ to the guiding nucleotide, the first phosphodiester bond 5′ to the guiding nucleotide, the second phosphodiester bond 5′ to the guiding nucleotide, or the third phosphodiester bond 5′ to the guiding nucleotide, respectively. Concurrently, the cleavage of nucleic acid strand by the endonuclease leaves the remaining nucleic acid strand having a free 3′-hydroxy group which can be used as a new, or a usable, initiator for another round of nucleic acid synthesis.

Furthermore, a thermophilic endonuclease can be utilized recognize the position of guiding nucleotide in the nascent nucleic acid strand and to perform a site-specific nucleic acid cleavage. Due to the thermophilic enzyme's intrinsic thermotolerant property, a thermostable endonuclease may catalyze the nucleic acid cleavage under a wide variety of reaction conditions, such as an elevated reaction temperature. In some embodiments, the endonuclease in the present disclosure is an endonuclease V, an endonuclease Q, NucS endonuclease, or EndoMS endonuclease.

Since the method provided herein may use non-limiting nucleic acid strands for cleavage and the user may obtain ready-to-use starting materials under various scenarios, also provides herein is a method of retrieving a predetermined sequence and length of polynucleotide enzymatically, which comprises the steps of: providing a synthetic nucleic acids containing a devised guiding nucleotide to be recognized by a selected endonuclease specifically; and subjecting the endonuclease to cleave the synthetic nucleic acids according to the position of the guiding nucleotide in the nucleic acid to release a predetermined, or desired, sequence and length of polynucleotide. As described previously, the endonuclease specifically recognizes the position of guiding nucleotide in the nucleic acids and preferably cleaves at the second phosphodiester bond 3′ to the guiding nucleotide, the first phosphodiester bond 5′ to the guiding nucleotide, the second phosphodiester bond 5′ to the guiding nucleotide, or the third phosphodiester bond 5′ to the guiding nucleotide, respectively, to obtain the predetermined polynucleotide.

Based on the method disclosed herein, a kit of synthesizing and retrieving a predetermined polynucleotide is provided herein. Under scenario of enzymatic nucleic acid synthesis, the kit may comprise: an initiator having a 3′-terminal nucleotide with a free 3′-hydroxyl group: a polymerase for incorporating nucleotide monomers, which comprise a guiding nucleotide monomer, to the initiator to elongate a nucleic acid strand from the free 3′-hydroxyl group, such that the guiding nucleotide is incorporated in the designated position of newly synthesized nucleic acid strand; and an endonuclease recognizes the guiding nucleotide in the newly synthesized nucleic acid strand and cleave the nucleic acid strand according to the position of the guiding nucleotide to release a the predetermined, or desired, sequence and length of polynucleotide, so that the remaining nucleic acid strand has a new free 3′-hydroxyl group and readily serves as a new initiator for another round of nucleic acid synthesis. Under scenario of enzymatic polynucleotide retrieval, the kit may comprise: a synthetic polynucleotide having a designated guiding nucleotide; and an endonuclease for recognizing the guiding nucleotide and cleaving the synthetic polynucleotide to release a predetermined, or desired, sequence and length of polynucleotide. The endonuclease recognizes the guiding nucleotide and cleaves at the specific phosphodiester bond according to the position of the guiding nucleotide in the synthetic polynucleotide, so that the predetermined, or desired, sequence and length of polynucleotide is obtained. Under both scenarios, the properties of site specificity of the endonuclease included in the kits have been described previously.

As exemplified in more detail below, the method and kit provided herein utilizes a Pfu Endo V, Pfu or Bpu Endo Q, Bsu or Tba Endo V, and a nucleic acid initiator or primer/template duplex to generate a nucleic acid strand containing a different, unique guiding nucleotide for a site-specific recognition and the phosphodiester bond cleavage at common enzymatic reaction temperature, such as ambient temperature (e.g., 10° C. to 40° C.), 37° C., or an elevated reaction temperature, such as 70° C. The following Examples and the corresponding results indicate that the method and kit provided herein can utilize a nucleic acid initiator or primer/template duplex to generate nucleic acid strand containing an unique guiding nucleotide for recognition and the site-specific nucleic acid cleavage to precisely release the predetermined, or desired, sequence and length of polynucleotide strand at a wide-range of reaction temperatures, which broadens the applications and utilities of enzymatic nucleic acid synthesis.

Endonucleases are enzymes that cleave nucleic acid (e.g., DNA or RNA) at phosphodiester bonds connecting the nucleotides and play an important role in maintaining biological functions such as nucleic acid mismatch repairs. Endonucleases are also utilized for applications in DNA manipulation. For example, the T7 endonuclease I is widely used in mismatch repair and genome editing (e.g., mutation and deletion). The enzyme recognizes the DNA mismatch and cleaves the first, second, or third phosphodiester bond 5′ to the mismatch position. E. coli Endonuclease V (Eco Endo V) is another enzyme that can specifically recognize DNA position for strand cleavage. The E. coli Endo V recognizes the deoxyinosine (dI) lesion in DNA and cleaves the second phosphodiester bond at the 3′-side of the dI lesion and generates a DNA strand break. As described above, E. coli Endo V has been used in the de novo enzymatic DNA synthesis to cleave the phosphodiester bond linkage between the initiator and the newly synthesized DNA. However, the narrow substrate specificity and limited thermal tolerance of E. coli Endo V restricts its utilization and broad applications in emerging enzymatic DNA synthesis.

Other endonucleases, such as endonuclease V derived from Pyrococcus furiosus (Pfu) or Thermococcus barophilus (Tba), endonuclease Q derived from Pyrococcus furiosus (Pfu), Methanosarcina acetivorans (Mac) and Bacillus pumilus (Bpu), NucS endonuclease derived from Pyrococcus abyssi (Pab) and Thermococcus gammatolerans (Tga), and EndoMS endonuclease from Thermococcus kodakarensis (Tko) have a broader substrate spectrum than E. coli Endo V. These endonucleases recognize various deaminated or oxidated bases, such as deoxyuridine or deoxyinosine in nucleic acids. In addition, these endonucleases may also recognize apurinic/apyrimidinic lesion in DNA, such as an abasic site or a dSpacer (also known as abasic furan) which is structurally similar to an abasic site.

Furthermore, these groups of endonucleases are more thermotolerant. Pfu Endo V, Tba Endo V and Pfu Endo Q, Mac Endo Q, Pab NucS, Tko EndoMS, and Tga NucS can cleave the DNA strand at a much broader reaction temperatures than E. coli Endo V, such as temperatures ranging from 10° C. to 100° C., or from 20° C. to 30° C., from 30° C. to 40° C., from 40° C. to 50° C., from 50° C. to 60° C., from 60° C. to 70° C., from 70° C. to 80° C., from 80° C. to 90° C., from 90° C. to 100° C. as demonstrated in the exemplary results. The method and kit disclosed herein provides an improved endonuclease-based nucleic acid strand cleavage approach compared to the conventional art, utilizing a nucleic acid initiator or primer, which includes a different type of deaminated or oxidated bases, such as deoxyuridine or deoxyinosine, for a site-specific recognition and cleavage of nucleic acid strand. Additionally, the method and kit disclosed herein provide an improved thermotolerant endonuclease based nucleic acid cleavage approach that can perform at a wider range of reaction temperatures, which broaden the applications and utilities of enzymatic nucleic acid synthesis. Furthermore, after the release of the predetermined, or desired, sequence and length of nucleic acid strand, or polynucleotide chain, the nucleic acid initiator or primer can be regenerated with a normal 3′-hydroxyl group. Hence, the nucleic acid initiator, or a primer, can be reused for new rounds of enzymatic nucleic acid synthesis.

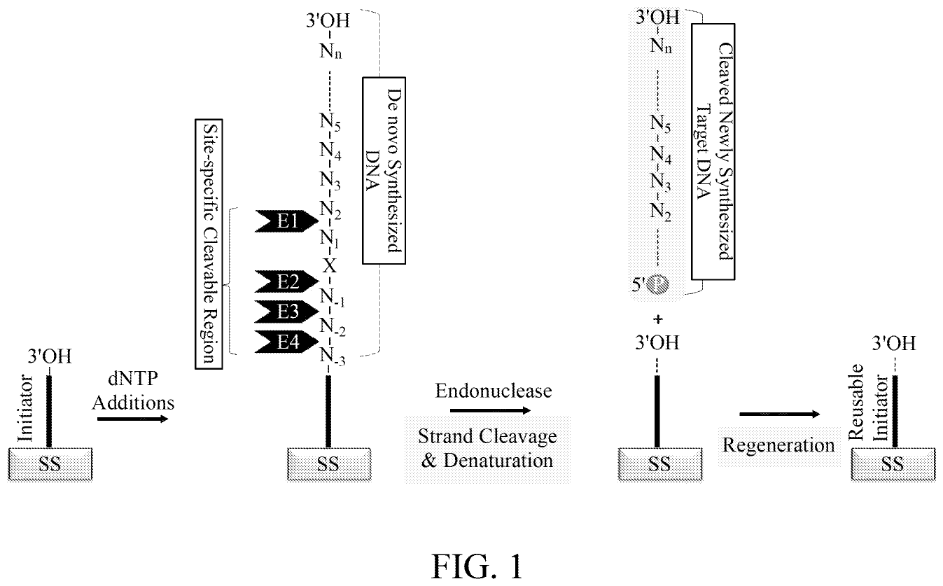

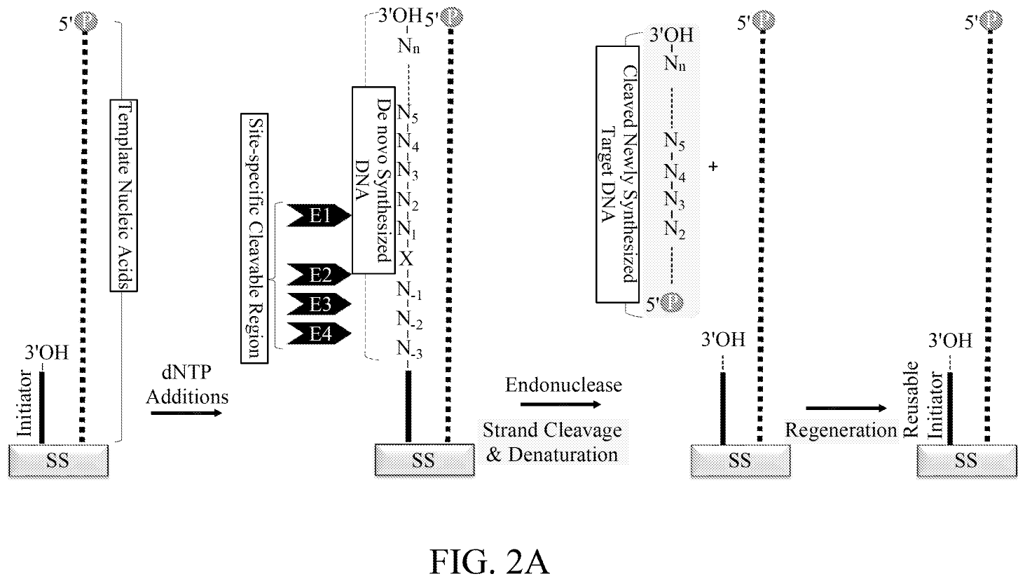

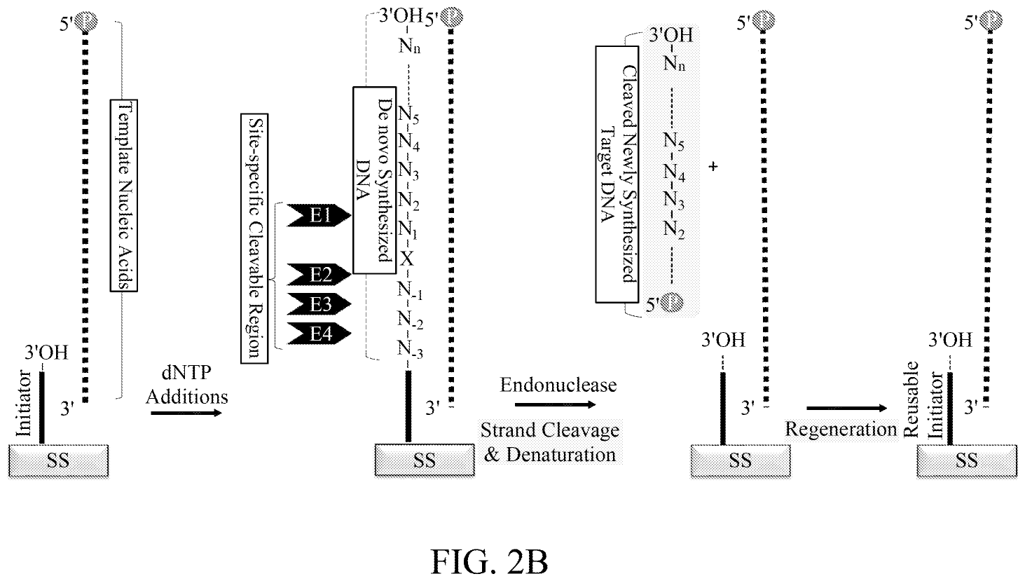

FIGS. 1, 2A, and 2B show the exemplary schemes of the present disclosure in a template-independent (FIG. 1) and a template-directed/template-dependent (FIGS. 2A and 2B) nucleic acid synthesis. As shown in FIGS. 1, 2A, and 2B, an initiator (ssDNA) attached to a solid support is provided. By contrast to the template-independent nucleic acid synthesis, the template-directed/template-dependent nucleic acid synthesis requires nucleic acids template attached to the solid support (FIG. 2A) or hybridized to the initiator by annealing method or other known hybridization techniques (FIG. 2B). In some embodiments, under the templated-dependent scenario, the primer is used in combination with a template to generate a primer/template duplex (P/T duplex) for synthesizing the DNA. In the exemplary schemes, the initiator or primer is elongated by a template-independent or template-dependent polymerase, such as a B-family DNA polymerase, with nucleotide monomers (N) containing a normal 3′-hydroxyl group or a 3′-blockage chemical moiety to synthesize the oligonucleotide/polynucleotide chain with desired sequence (N₋₃ to Nₙ). The sequence of the nucleic acid strand is configured to comprise at least one guiding nucleotide (X) having a predetermined nucleobase or a predetermined apurinic/apyrimidinic (AP) lesion to be recognized by a selected endonuclease. In some embodiments, the predetermined nucleobase may be a deoxyuridine or a deoxyinosine, and the apurinic/apyrimidinic lesion may be an abasic site or a dSpacer, and each recognizable deoxynucleotide residue can be specifically recognized by different types of corresponding endonucleases, respectively. As such, the present method and kit provide an approach to incorporate nucleotide monomers (including canonical and non-canonical nucleotide) to the initiator by the polymerase to synthesize and retrieve a custom-made polynucleotide chain which is site-specifically cleavable according to the needs of the user.

The inventor has surprisingly discovered that the nucleic acid cleavage activity of the endonucleases disclosed herein can recognize a guiding nucleotide in the site-specific cleavable region. After sufficient cycles of nucleotide addition/incorporation to generate the desired nucleic acid strand, the endonuclease-based cleavage reaction is introduced to cleave and/or denature the nucleic acid strand or polynucleotide chain that comprises site-specific cleavable region near the guiding nucleotide. With respect to the guiding nucleotide (X) for endonuclease-based recognition in the site-specific cleavable region, a corresponding endonuclease is introduced for the nucleic acid cleavage. In at least one embodiment of the present disclosure, the nucleic acid strand having a site-specific cleavable region containing deoxyinosine or deoxyuridine, respectively, may be treated with an endonuclease Q derived from Pyrococcus furiosus (Pfu), Methanosarcina acetivorans (Mac), or Bacillus pumilus (Bpu), endonuclease V derived from Bacillus subtilis (Bsu), Escherichia (Eco), Pyrococcus furiosus (Pfu), or Thermococcus barophilus (Tba), NucS endonuclease derived from Thermococcus gammatolerans (Tga) or Pyrococcus abyssi (Pab), or EndoMS endonuclease derived from Thermococcus kodakarensis (Tko) to release the predetermined elongated nucleic acid strand. In the exemplary schemes, the endonucleases E₁ to E₄ recognize the guiding nucleotide (X) and cleave phosphodiester bonds between N₂ and N₁, between X and N. 1, between N₋₁, and N₋₂, and between N₋₂ and N₋₃, respectively. In some embodiment, X may be deoxyinosine or deoxyuridine: E₁ may be Bsu EndoV, Eco EndoV, Pfu Endo V, or Tba Endo V: E₂ may be Pfu EndoQ, Mac EndoQ, Bpu Endo Q: E₃ may be Tga NucS; and E₄: may be Tko EndoMS, but the present disclosure is not limited thereto. In addition, the thermotolerant characteristic of the endonucleases disclosed herein enables a nucleic acid strand cleavage reaction at a wide range of reaction temperatures. In combination of the favorable properties of the endonucleases disclosed herein, the present methods of disclosure can be utilized for various conditions and applications of enzymatic nucleic acid synthesis. The site-specific cleavage of the newly synthesized nucleic acid strands or polynucleotide chain by the endonuclease not only releases the desired polynucleotide fragment, but also regenerates a new free 3′-hydroxyl group at 3′ end of the remaining nucleic acid strand to be reused for a new round of synthesis reaction, so when combined with a proper design and setting of nucleotide monomer additions, the enzymatic synthesis can be efficiently cycled without interruption of additional enzyme treatment (e.g., dephosphorylation by the phosphatase). Therefore, the present disclosure provides a precise, efficient, cost-effective, and thermotolerant approach to retrieve the predetermined nucleic acid strand or polynucleotide chain, and to concurrently regenerate the reusable nucleic acid initiator with a free hydroxyl group in one step for accelerating the user-desired nucleic acid synthesis.

### EXAMPLES

The present disclosure is further described by means of the following examples. However, these examples are only illustrative of the disclosure, and in no way limit the scope and meaning of the present disclosure. Indeed, many modifications and variations of the present disclosure will be apparent to those skilled in the art upon reading this specification, and can be made without departing from its scope.

### A. Nucleic Acid Synthesis by a Nucleic Acid Polymerase to Incorporate Nucleotide Monomers to an Initiator

The following synthetic polynucleotide initiator, FAM-45-mer DNA initiator, was used for the template-independent DNA synthesis in this example.

FAM-45-mer DNA initiator:
(SEQ ID NO: 1)
5′-CTCGGCCTGGCACAGGTCCGTTCAGTGCTGCGGCGACCACC
GAGG-3′.

This single-stranded 45-mer polynucleotide initiator is modified with a biotin group at the 5′-end and an internal fluorescein amidite (FAM) dye at the 23^{rd} thymidine base (the underline T) and has a free 3′-hydroxyl group at the 3′ end thereof. In addition, the 5′ end of the initiator was immobilized to Dynabeads™ M-280 Streptavidin beads. Nevertheless, in other embodiments, the initiator may be internally labeledwith a Hexachloro-fluorescein (HEX) or other fluorescent reporter dyes as disclosed herein, and the initiator may be free form or immobilized via 5′-end by other solid supports other than Dynabeads™ M-280 Streptavidin beads as disclosed herein.

Further, the template-independent nucleic acid synthesis reaction was performed using a B-family DNA polymerase (1 μM) to incorporate a linking 3-O-(azidomethyl)-2′-deoxyuridine triphosphate (100 μM) to the 3′ end of the initiator for 15 minutes. To demonstrate the template-independent nucleic acid synthesis right after the incorporation of the deoxyuridine monophosphate (dUMP) at the 3′-end of the synthesis initiator, the B-family DNA polymerase (1 μM) was used to stepwise incorporate a 3′-O-(azidomethyl)-2′-deoxyguanosine triphosphate (100 μM) or dNTP mixture (dATP, dCTP, dGTP, and dTTP) (100 μM) to the initiator containing the deoxyuridine (U) at the 3′ terminus. The synthesis reaction was initiated by addition of 10 mM manganese cations and then incubated at 75° C. for 15 minutes. The reaction was stopped by adding 10 μL of a 2× quench solution (95% deionized formamide and 25 mM EDTA) and subjected to the heat denaturation at 98° C. for 10 minutes. The reaction products were analyzed by a 15% denaturing urea-polyacrylamide gel, and the gel results were visualized by Amersham Typhoon Imager, GE Healthcare Life Sciences (Marlborough, MA., United States).

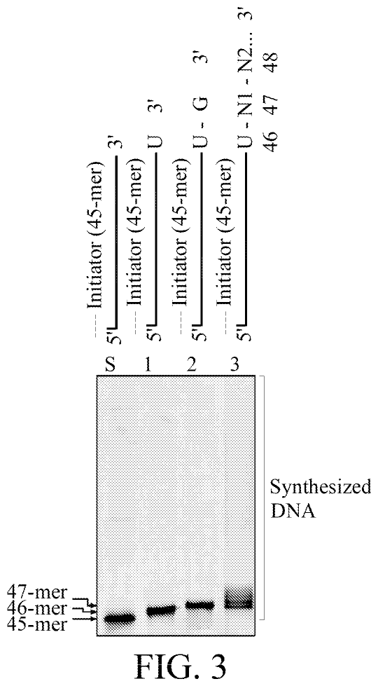

As illustrated in FIG. 3, lane S shows the initiator only, and lane 1 shows that the deoxyuridine triphosphate (dUTP) was efficiently incorporated to the 3′-end of the initiator by the B-family DNA polymerase. Further, lanes 2 and 3 illustrate that the B-family DNA polymerase can incorporate deoxyguanosine triphosphate (dGTP) and dNTP (N₁, N₂, etc), respectfully, right after the deoxyuridine (U) at the 3′ end of the initiator. The polynucleotide samples of lane 1 and lane 2 have SEQ ID NO: 2 and SEQ ID NO: 3, respectively.

Therefore, the example demonstrates a method for incorporating canonical (e.g., dNTP) or non-canonical nucleotide (e.g., deoxyuridine or deoxyguanosine triphosphate) to the initiator by the polymerase to synthesize a custom-made polynucleotide chain containing at least one exemplary guiding nucleotide located at the desired site to be recognized by the endonuclease according to the needs of the user.

Although the above example demonstrated the result of template-independent nucleic acid synthesis, the initiator may also be designed for a template-dependent nucleic acid synthesis in other embodiments and achieve similar results. For instance, the complementary template nucleic acids of the initiator may be attached to the solid support (FIG. 2A) or hybridized to the initiator to form a duplex or double strand (FIG. 2B).

### B. Cleavage or Denaturation of the Newly Synthesized Polynucleotide by an Endonuclease to Release a Desired Nucleic Acid Fragment Thereof and Regenerate an Initiator for the Next-Round of Nucleic Acid Synthesis

To demonstrate the feasibility of cleaving/denaturing the newly synthesized polynucleotide and regenerating the synthesis initiator by virtue of an endonuclease, the single-stranded 38-mer polynucleotides labelled with Hex dye, containing an deoxyuridine (U) or an deoxyinosine (I), designated as Hex-Top-U38-mer and Hex-Top-138-mer, respectively, were synthesized using the method mentioned in Section A above and served as a representative nucleic acid product that contains an initiator and newly synthesized polynucleotide by the template-independent nucleic acid synthesis. In addition, to exemplify a nucleic acid product that contains an initiator and newly synthesized polynucleotide by the template-dependent nucleic acid synthesis, the Hex-Top-U38-mer and Hex-Top-138-mer were hybridized with a complementary single-stranded 38-mer nucleic acid (Bot-A38-mer) at a molar ratio of 1:1.5 in the 1× TE buffer containing 100 mM of NaCl. The DNA annealing reaction was performed in the Bio-Rad thermal cycler machine by heating up the sample mixture to 95° C. for 3 minutes and gradually cooling it down (5° C./30 seconds) to 4° C. to from a duplex and double-stranded 38-mer nucleic acid. These two nucleic acid products serve as the DNA substrate in the following examples.

The polynucleotide sequences, buffers, and solutions of the examples are listed in the tables below. The Hex-Top-U38-mer and Hex-Top-138-mer has a Hexachloro-fluorescein (HEX) labeled at the 5′ end thereof and a free 3′-hydroxyl group at the 3′ terminus thereof, but the present disclosure is not limited thereto. In other embodiments, the 5′ end of the initiator may be labeled by fluorescein amidite (FAM) or other fluorescent reporter dyes disclosed herein. In addition, the initiator may be in a free form or immobilized to the solid support disclosed herein in different embodiments.

TABLE 1
Polynucleotide sequences

| Polynucleotide | Sequence |
|---|---|
| Hex-Top-U38-mer | 5′-CAGGGATCCGTGAAGCTATCCUGCGTC |
| | TAGGACTAAGC-3′ (SEQ ID NO: 4) |
| Hex-Top-I38-mer | 5′-CAGGGATCCGTGAAGCTATCCIGCGTC |
| | TAGGACTAAGC-3′ (SEQ ID NO: 5) |
| Bot-A38-mer | 5′-GCTTAGTCCTAGACGCAGGATAGCTTC |
| | ACGGATCCCTG-3′ (SEQ ID NO: 6) |

TABLE 2
Buffers and solutions

| Buffers and solutions | Components |
|---|---|
| 1 × TE buffer | 10 mM Tris-HCl (pH 8.0) |
| | 1 mM EDTA |
| Enzyme reaction buffer | 50 mM potassium acetate |
| | 20 mM Tris-acetate |
| | 10 mM magnesium acetate |
| | 10 mM DTT (pH 7.9 at 25° C.) |
| 2 × quench solution | 95% formamide |
| | 25 mM EDTA |

To test whether the newly synthesized polynucleotide derived from the template-independent or template-directed nucleic acid synthesis that comprises a guiding nucleotide with a predetermined nucleobase or apurinic/apyrimidinic (AP) lesion can be recognized and cleaved to regenerate an initiator for the next-round of nucleic acid synthesis, the following experimental procedures were conducted.

### Example 1: Recognition of Deoxyinosine and DNA-Strand Cleavage by Pfu Endo V at Two Different Reaction Temperatures

The sample groups include (1) only the DNA substrate(S) that serves as a negative control: (2) an in-house E. coli endonuclease V (Eco EndoV) (SEQ ID NO: 7), which can be referred to US 2021/0254114A1; (3) a Pyrococcus furiosus endonuclease V (Pfu Endo V) (SEQ ID NO: 8): (4) an E. coli endonuclease V obtained from New England BioLabs, Ipswich, MA, (C1); and (5) the enzyme mixture containing human alkyladenine DNA glycosylase (hAAG) and EndoVIII (C2) that serves as a positive control for the deoxyinosine excision and the DNA strand cleavage at the guiding nucleotide position in the nucleic acid product thereof. The sample mixtures (10 μl) containing a 100 nM of a single-stranded Hex-Top-138-mer DNA substrate or a double-stranded Hex-Top-138-mer/Bot-A38-mer DNA substrate were incubated with 400 nM endonuclease in each sample group in the enzyme reaction buffer. The sample mixtures were incubated at 37° C. or 70° C., respectively, for 20 minutes. Each enzyme reaction was stopped by the addition of equal volume (10 μL) of 2× quench solution.

The total 20 μL of sample were denatured at 95° C. for 10 min, and 4 μL of each sample mixture were analyzed by 20% denaturing polyacrylamide gel electrophoresis containing 8 M urea in the 1× TBE buffer (90 mM Tris-base, 90 mM boric acid, and 2 mM EDTA). The results of the gel were then visualized by Amersham Typhoon scanner (Cytiva, Marlborough, MA).

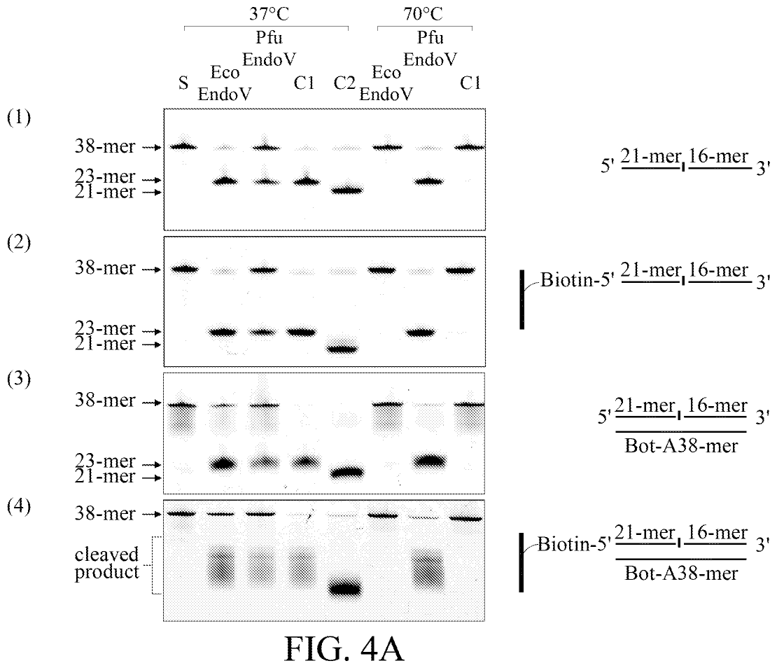

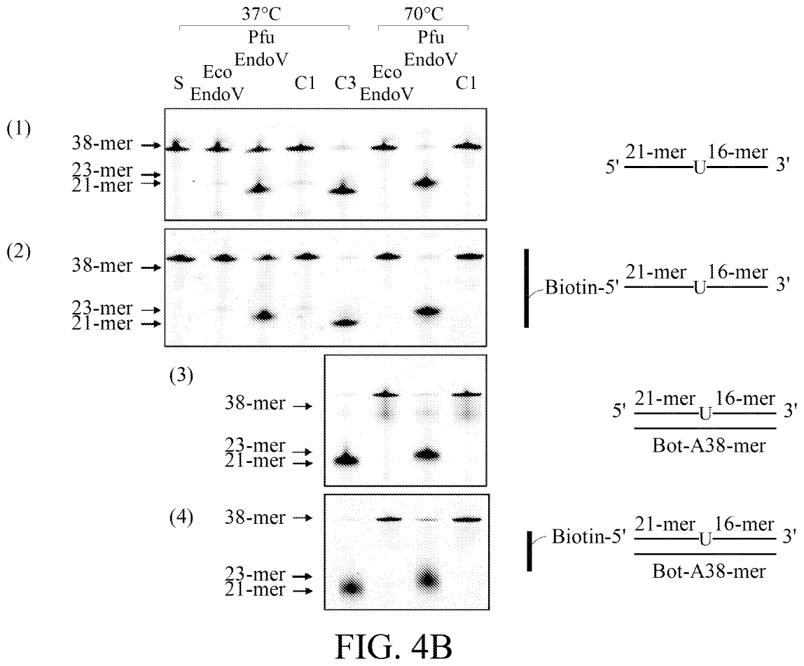

As illustrated in FIG. 4A and Table 3 below, Eco EndoV, Pfu Endo V, and C1 efficiently recognized deoxyinosine (I) and cleaved the phosphodiester bond between the first nucleotide (G) and the second nucleotide (C) starting from the deoxyinosine (I) toward 3′ end, or downstream, of the DNA at the reaction temperature of 37° C., thereby releasing a 15-mer single, or double stranded, DNA and the remaining 23-mer single, or double-stranded, DNA with a free 3′-hydroxyl group at the terminus, which can readily serve as a new (or a reusable) initiator for the next-round of nucleic acid synthesis. By contrast, C2 only performed the deoxyinosine (I) excision and DNA strand cleavage at the reaction temperature of 37° C. to release 15-mer single, or double-stranded, DNA and failed to generate a free 3′-hydroxyl group at the 3′ terminus of the remaining 21-mer single, or double-stranded, DNA, which cannot be used for the new round of nucleic acid synthesis. Also, it was noted that only Pfu Endo V efficiently cleaved or denatured the DNA strand as mentioned above at the reaction temperature of 70° C.

TABLE 3
Site-specific recognition and cleavage
of Hex-Top-I38-mer by Pfu EndoV

| | Site-specific recognition and |
|---|---|
| | cleavage of Hex-Top-I38-mer DNA |
| | (SEQ ID NO: 5) (↑ refers to |
| Enzyme | the cleavage site of the enzyme) |
| Eco Endo V, | 5′-CAGGGATCCGTGAAGCTATCCIG ↑ CGT |
| Pfu Endo V, | CTAGGACTAAGC-3′ |
| C1 | |
| C2 | 5′-CAGGGATCCGTGAAGCTATCC ↑ I ↑ G |
| | CGTCTAGGACTAAGC-3′ |

### Example 2: Deoxyuridine Recognition and DNA-Strand Cleavage by Pfu Endo V at Two Different Reaction Temperatures

The sample groups include (1) the only DNA substrate(S) that serves as a negative control; (2) an in-house E. coli endonuclease V (Eco EndoV), which can be referred to US 2021/0254114A1; (3) a Pyrococcus furiosus endonuclease V (Pfu Endo V); (4) an E. coli endonuclease V obtained from New England BioLabs, Ipswich, MA, (C1); and (5) an uracil-specific excision reagent (C3) from New England Biolabs (Cat. #M5505S, Ipswich, MA) that serves as a positive control. The sample mixtures (10 μl) contain a 100 nM of a single-stranded Hex-Top-U38-mer DNA substrate or a double-stranded Hex-Top-U38-mer/Bot-A38-mer DNA substrate. The samples were processed and analyzed as described in Example 1, and details thereof are omitted herein for the sake of brevity.

As illustrated in FIG. 4B and Table 4 below, Pfu Endo V efficiently recognized deoxyuridine (U) and cleaved the phosphodiester bond between the first nucleotide (G) and the second nucleotide (C) starting from the deoxyuridine (U) toward 3′ end, or downstream, of the DNA at the reaction temperature of 37° C. or 70° C., thereby releasing a 15-mer single, or double-stranded, DNA and the remaining 23-mer single, or double-stranded, DNA with a free 3′-hydroxyl group at the terminus, which can readily serve as a new, or reusable, initiator for the next-round of nucleic acid synthesis. By contrast, Eco EndoV and C1 failed to demonstrate such site-specific cleavage or denaturation. In addition, C3 only cleaved the phosphodiester bond between the deoxyuridine (U) and the first nucleotide (G) starting from the deoxyuridine (U) toward 5′ end, or upstream, of the DNA at the reaction temperature of 37° C. to release 17-mer single, or double-stranded, DNA and failed to generate a free 3′-hydroxyl group at the 3′ terminus of the remaining 21-mer single, or double-stranded, DNA, which cannot be used for the new round of nucleic acid synthesis.

TABLE 4
Site-specific recognition and cleavage
of Hex-Top-U38-mer by Pfu Endo V

| | Site-specific cleavage of |
|---|---|
| | Hex-Top-U38-mer DNA |
| | (SEQ ID NO: 4) (↑ refers to |
| Enzyme | the cleavage site of the enzyme) |
| Pfu Endo V | 5′-CAGGGATCCGTGAAGCTATCCUG ↑ CGT |
| | CTAGGACTAAGC-3′ |
| C3 | 5′-CAGGGATCCGTGAAGCTATCC ↑ UGCGT |
| | CTAGGACTAAGC-3′ |

### Example 3: Deoxyinosine Recognition and DNA-Strand Cleavage by Pfu Endo Q at Two Different Reaction Temperatures

The sample groups include (1) a Pyrococcus furiosus endonuclease Q (Pfu Endo Q) (SEQ ID NO: 9): (2) an E. coli endonuclease V obtained from New England BioLabs, Ipswich, MA, (C1); and (3) the enzyme mixture containing human alkyladenine DNA glycosylase (hAAG) and EndoVIII (C2) that serves as a positive control for the deoxyinosine excision and the DNA strand cleavage at the guiding nucleotide position thereof. The sample mixtures (10 μl) contain a 100 nM of a single-stranded Hex-Top-138-mer DNA substrate or a double-stranded Hex-Top-138-mer/Bot-A38-mer DNA substrate. The samples were processed and analyzed as described in Example 1, and details thereof are omitted herein for the sake of brevity.

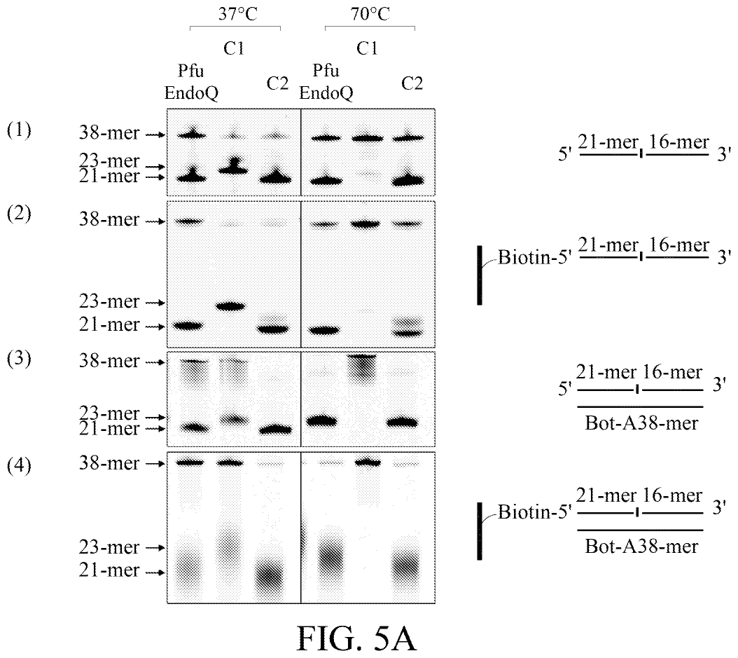

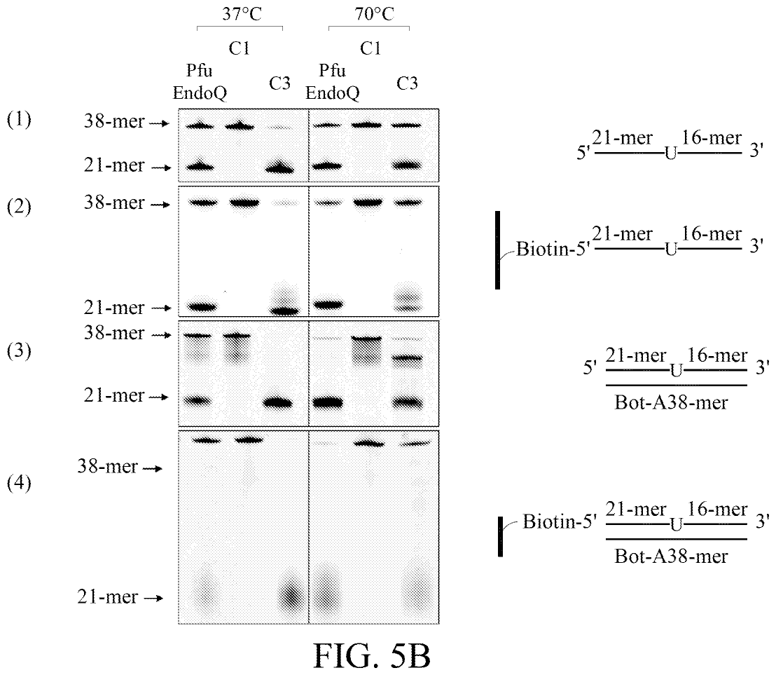

As illustrated in FIG. 5A and Table 5 below, Pfu Endo Q efficiently recognized deoxyinosine (I) and cleaved the phosphodiester bond between the deoxyinosine (I) and the first nucleotide (C) starting from the deoxyinosine (I) toward 5′ end of the DNA at the reaction temperature of 37° C. or 70° C., thereby releasing a 17-mer single or double stranded DNA and the remaining 21-mer single, or double-stranded, DNA with a free 3′-hydroxyl group at the 3′-terminus, which can readily serve as a new, or reusable, initiator for the next-round of nucleic acid synthesis. Also, C1 can recognized deoxyinosine (I) and cleaved the phosphodiester bond between the first nucleotide (G) and the second nucleotide (G) starting from the deoxyinosine (I) toward 3′ end of the DNA at the reaction temperature of 37° C., thereby releasing a 15-mer single, or double-stranded, DNA and the remaining 23-mer single, or double-stranded, DNA with a free 3′-hydroxyl group at the 3′-terminus, which can readily serve as a new, or reusable, initiator for the next-round of nucleic acid synthesis. However, CI failed to demonstrate the site-specific cleavage on the DNA at the reaction temperature of 70° C. In addition, C2 only performed the deoxyinosine (I) excision and DNA strand cleavage to release 15-mer single, or double-stranded DNA, and failed to generate a free 3′-hydroxyl group at the 3′ terminus of the remaining 21-mer single, or double-stranded, DNA, which cannot be used for the new round of nucleic acid synthesis.

TABLE 5
Site-specific recognition and cleavage
of Hex-Top-I38-mer by Pfu EndoQ

| | Site-specific cleavage |
|---|---|
| | of Hex-Top-I38-mer DNA |
| | (SEQ ID NO: 5) (↑ refers to |
| Enzyme | the cleavage site of the enzyme) |
| Pfu Endo Q | 5′-CAGGGATCCGTGAAGCTATCC ↑ IGCGT |
| | CTAGGACTAAGC-3′ |
| C1 | 5′-CAGGGATCCGTGAAGCTATCCIG ↑ CGT |
| | CTAGGACTAAGC-3′ |
| C2 | 5′-CAGGGATCCGTGAAGCTATCC ↑ I ↑ G |
| | CGTCTAGGACTAAGC-3′ |

### Example 4: The Deoxyuridine Recognition and DNA-Strand Cleavage by Pfu Endo Q at Two Different Reaction Temperatures

The sample groups include (1) a Pyrococcus furiosus endonuclease Q (Pfu Endo Q); (2) an E. coli endonuclease V obtained from New England BioLabs, Ipswich, MA, (C1); and (3) an uracil-specific excision reagent (C3) from New England Biolabs (Cat. #M5505S, Ipswich, MA) that serves as a positive control. The sample mixtures (10 μl) contain a 100 nM of a single-stranded Hex-Top-U38-mer DNA substrate or a double-stranded Hex-Top-U38-mer/Bot-A38-mer DNA substrate. The samples were processed and analyzed as described in Example 1, and details thereof are omitted herein for the sake of brevity.

As illustrated in FIG. 5B and Table 6 below, Pfu Endo Q efficiently recognized deoxyuridine (U) and cleaved the phosphodiester bond between the deoxyuridine (U) and the first nucleotide (C) starting from the deoxyinosine (I) toward 5′ end of the DNA at reaction temperature of 37° C. or 70° C., thereby releasing a 17-mer single, or double-stranded, DNA and the remaining 21-mer single, or double-stranded, DNA with a free 3′-hydroxyl group at the 3′-terminus, which can readily serve as a new, or reusable, initiator for the next-round of nucleic acid synthesis. C3 exhibited the similarly site-specific cleavage on the DNA as Pfu Endo Q to release17-mer single, or double-stranded, DNA, while C3 failed to generate a free 3′-hydroxyl group at the 3′ terminus of the remaining 21-mer single, or double-stranded, DNA and cannot be used for the new round of nucleic acid synthesis. By contrast, C1 failed to demonstrate the site-specific cleavage on the DNA at the reaction temperature of 37° C. or 70° C.

TABLE 6
Site-specific recognition and cleavage
of Hex-Top-U38-mer by Pfu EndoQ

| | Site-specific cleavage |
|---|---|
| | of Hex-Top-U38-mer DNA |
| | (SEQ ID NO: 4) (↑ refers to |
| Enzyme | the cleavage site of the enzyme) |
| Pfu Endo Q, | 5′-CAGGGATCCGTGAAGCTATCC ↑ UGCGT |
| C3 | CTAGGACTAAGC-3′ |

### Example 5: Deoxyinosine Recognition and DNA-Strand Cleavage by Tba Endo V at Two Different Reaction Temperatures

The sample groups include (1) only the DNA substrate(S) that serves as a negative control; (2) an in-house E. coli endonuclease V (Eco EndoV), which can be referred to US 2021/0254114A1; (3) a Thermococcus barophilus endonuclease V (Tba Endo V) (SEQ ID NO: 10); (4) the enzyme mixture containing hAAG and Endo VIII (C2) that serves as a positive control for the deoxyinosine excision and the DNA strand cleavage at the guiding nucleotide position thereof. The sample mixtures (10 μl) contain a 100 nM of a single-stranded Hex-Top-138-mer DNA substrate or a double-stranded Hex-Top-138-mer/Bot-A38-mer DNA substrate. The samples were processed and analyzed as described in Example 1, and details thereof are omitted herein for the sake of brevity.

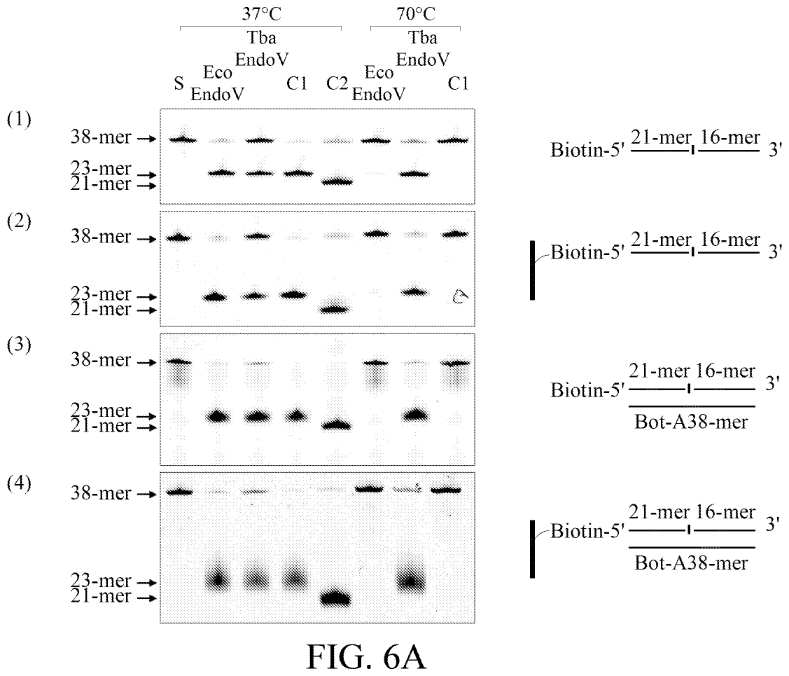

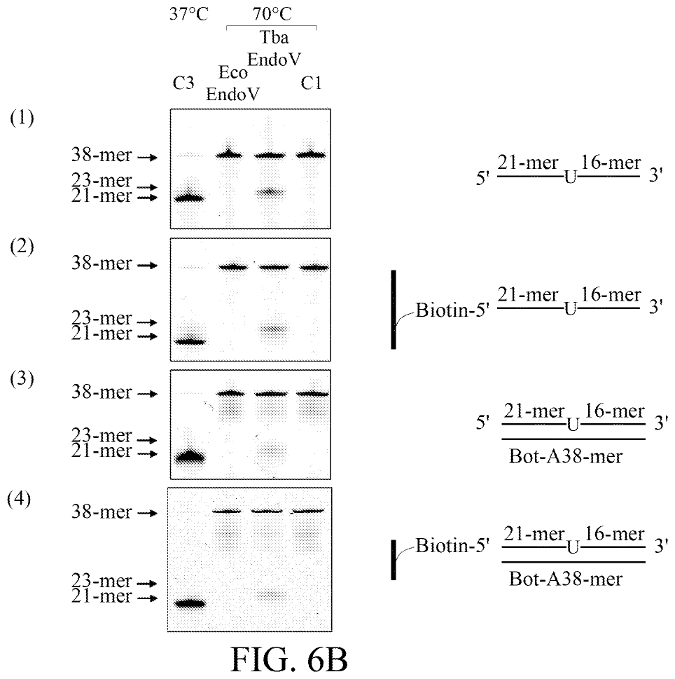

As illustrated in FIG. 6A and Table 7 below, Eco EndoV, Tba Endo V, and C1 efficiently recognized deoxyinosine (I) and cleaved the phosphodiester bond between the first nucleotide (G) and the second nucleotide (C) starting from the deoxyinosine (I) toward 3′ end of the DNA at the reaction temperature of 37° C., thereby releasing a 15-mer single, or double-stranded, DNA and the remaining 23-mer single, or double-stranded, DNA with a free 3′-hydroxyl group at the 3′-terminus, which can readily serve as a new, or reusable, initiator for the next-round of nucleic acid synthesis. By contrast, C2 only performed deoxyinosine (I) excision and DNA strand cleavage at the reaction temperature to release 15-mer single, or double-stranded, DNA, and failed to generate a free 3′-hydroxyl group at the 3′ terminus of the remaining 21-mer single, or double-stranded, DNA and cannot be used for the new round of nucleic acid synthesis. Also, it was noted that only Tba Endo V efficiently cleaved, or denatured, the DNA strand as mentioned above at the reaction temperature of 70° C. as compared with Eco EndoV and C1.

TABLE 7
Site-specific recognition and cleavage
of Hex-Top-I38-mer by Tba Endo V

| | Site-specific cleavage |
|---|---|
| | of Hex-Top-I38-mer DNA |
| | (SEQ ID NO: 5) (↑ refers to |
| Enzyme | the cleavage site of the enzyme) |
| Eco Endo V, | 5′-CAGGGATCCGTGAAGCTATCCIG ↑ CGT |
| Tba Endo V, | CTAGGACTAAGC-3′ |
| C1 | |
| C2 | 5′-CAGGGATCCGTGAAGCTATCC ↑ I ↑ G |
| | CGTCTAGGACTAAGC-3′ |

### Example 6: Deoxyuridine Recognition and DNA-Strand Cleavage by Tba Endo V at Two Different Reaction Temperatures

The sample groups include (1) only the DNA substrate(S) that serves as a negative control: (2) an in-house E. coli endonuclease V (Eco EndoV), which can be referred to US 2021/0254114A1: (3) a Thermococcus barophilus endonuclease V (Tba Endo V); (4) an uracil-specific excision reagent (C3) from New England Biolabs (Cat. #M5505S, Ipswich, MA) that serves as a positive control. The sample mixtures (10 μl) contain a 100 nM of a single-stranded Hex-Top-U38-mer DNA substrate or a double-stranded Hex-Top-U38-mer/Bot-A38-mer DNA substrate. The samples were processed and analyzed as described in Example 1, and details thereof are omitted herein for the sake of brevity.

As illustrated in FIG. 6B and Table 8 below, Tba Endo V efficiently recognized deoxyuridine (U) and cleaved the phosphodiester bond between the first nucleotide (G) and the second nucleotide (C) starting from the deoxyinosine (I) toward 3′ end of the DNA at reaction temperature of 70° C., thereby releasing a 15-mer single, or double stranded, DNA and the remaining 23-mer single, or double-stranded, DNA with a free 3′-hydroxyl group at the 3′-terminus, which can readily serve as a new, or reusable, initiator for the next-round of nucleic acid synthesis. By contrast, Eco EndoV and C1 failed to demonstrate the site-specific cleavage on the DNA at the reaction temperature of 70° C. In addition, C3 exhibited the similarly site-specific cleavage on the DNA as Pfu Endo Q to release a17-mer single, or double-stranded, DNA: however, C3 failed to generate a free 3′-hydroxyl group at the 3′ terminus of the remaining 21-mer single, or double-stranded, DNA and cannot be used for the new round of nucleic acid synthesis.

TABLE 8
Site-specific recognition and cleavage
of Hex-Top-U38-mer by Pfu EndoQ

| | Site-specific cleavage |
|---|---|
| | of Hex-Top-U38-mer DNA |
| | (SEQ ID NO: 4) (↑ refers to |
| Enzyme | the cleavage site of the enzyme) |
| Tba Endo V, | 5′-CAGGGATCCGTGAAGCTATCCUG ↑ CGT |
| Eco Endo V, | CTAGGACTAAGC-3′ |
| C1 | |
| C3 | 5′-CAGGGATCCGTGAAGCTATCC ↑ UGCGT |
| | CTAGGACTAAGC-3′ |

The Tba Endo V shares comparable experimental results as Pfu Endo V and Q. As shown in the Examples 5 and 6 (referring to FIGS. 6A and 6B, respectively), Tba Endo V efficiently and site-specifically cleaved the DNA strand from a DNA initiator/primer containing either a deoxyinosine or a deoxyuridine. On the contrary, E. coli Endo V only cleaves the DNA strand from a DNA initiator/primer containing a deoxyinosine. Also, Tba Endo V efficiently and site-specifically cleaves the DNA strand from a DNA initiator/primer containing either a deoxyinosine or a deoxyuridine at both 37° C. and 70° C., while E. coli Endo V only cleaves the DNA strand at 37° C. Likewise, Tba Endo V also efficiently cleave the deoxyinosine- or deoxyuridine-containing primer-template DNA duplex. The results of hAAG plus Endo VIII and NEB USER herein, respectively, and the commercial NEB Eco Endo V enzymes confirm the relative positions of intact and cleaved DNA fragments in the gel generated by Tba EndoV cleavage.

As shown in the Examples 1 to 6 (referring to FIGS. 4A to 6B, respectively), Pfu Endo V, Pfu Endo Q, and Tba Endo V efficiently and site-specifically cleaved the DNA strand of the synthesized single, or double-stranded, DNA containing either a dexoyinosine or a dexoyuridine. On the contrary, Eco Endo V could only cleave the DNA strand from the DNA containing the dexoyinosine. Furthermore, Pfu Endo V, Pfu Endo Q, and Tba Endo V efficiently and site-specifically cleaves the DNA at both 37° C. and 70° C., while the E. coli Endo V only cleaves the DNA strand at 37° C. Additionally, the free form or immobilized form of the initiator did not affect the DNA cleavage activity of Pfu Endo V, Pfu Endo Q, and Tba Endo V. Also, although the positive control C2 and C3 could both cleave the DNA, they failed to generate a free 3′-hydroxyl group at the 3′ terminus of the remaining single, or double-stranded, DNA and cannot be used for the new round of nucleic acid synthesis. Given the foregoing, Pfu Endo V, Pfu Endo Q, and Tba Endo V can site-specifically recognize a guiding nucleotide, efficiently cleave the nucleic acid strand according to the position of the guiding nucleotide, and effectively regenerate an initiator with a free 3′-hydroxyl group at the 3′-terminus at a wide range of reaction temperatures including hyperthermal reaction temperatures.

### Example 7: Deoxyuridine Recognition and DNA-Strand Cleavage by Bsu Endo V

The sample groups include (1) only the DNA substrate(S) that serves as a negative control: (2) an in-house E. coli endonuclease V (Eco EndoV), which can be referred to US 2021/0254114A1: (3) a Bacillus subtilis endonuclease V (Bsu Endo V) (SEQ ID NO: 11): (4) an E. coli endonuclease V obtained from New England BioLabs, Ipswich, MA, (C1); and (5) an uracil-specific excision reagent (C3) from New England Biolabs (Cat. #M5505S, Ipswich, MA) that serves as a positive control. The sample mixtures (10 μl) contain a 100 nM of a single-stranded Hex-Top-U38-mer DNA substrate or a double-stranded Hex-Top-U38-mer/Bot-A38-mer DNA substrate. The samples were processed and analyzed as described in Example 1, and details thereof are omitted herein for the sake of brevity.

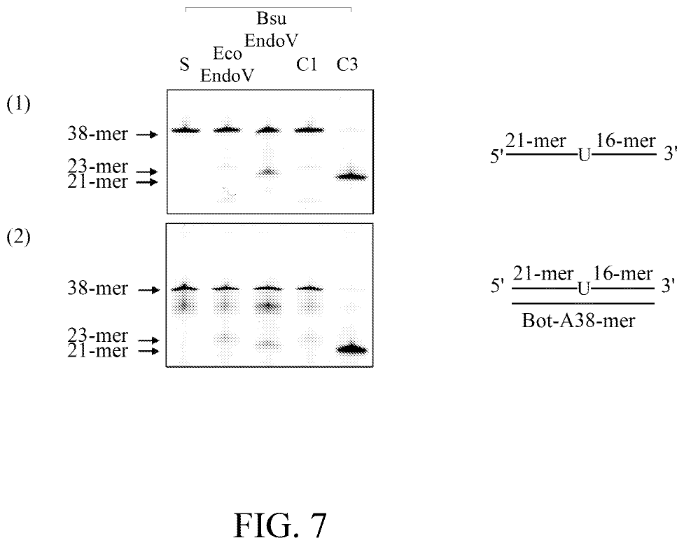

As illustrated in FIG. 7 and Table 9 below, Bsu Endo V efficiently recognized deoxyuridine (U) and cleaved the phosphodiester bond between the first nucleotide (G) and the second nucleotide (C) starting from the deoxyuridine (U) toward 3′ end of the DNA, thereby releasing a 15-mer single, or double-stranded, DNA and the remaining 23-mer single, or double-stranded, DNA with a free 3′-hydroxyl group at the 3′-terminus, which can readily serve as a new, or reusable, initiator for the next-round of nucleic acid synthesis. By contrast, Eco EndoV and C1 failed to demonstrate such site-specific cleavage or denaturation. In addition, C3 only cleaved the phosphodiester bond between the deoxyuridine (U) and the first nucleotide (G) starting from the deoxyuridine (U) toward 5′ end of the DNA to release a 17-mer single, or double-stranded, DNA and failed to generate a free 3′-hydroxyl group at the 3′ terminus of the remaining 21-mer single, or double-stranded, DNA and cannot be used for the new round of nucleic acid synthesis. Hence, Bsu Endo V can site-specifically recognize a guiding nucleotide, efficiently cleave the nucleic acid strand according to the position of the guiding nucleotide, and effectively regenerate an initiator with a free 3′-hydroxyl group at the 3′-terminus.

TABLE 9
Site-specific recognition and cleavage
of Hex-Top-U38-mer by Pfu Endo V

| | Site-specific cleavage |
|---|---|
| | of Hex-Top-U38-mer DNA |
| | (SEQ ID NO: 4) (↑ refers to |
| Enzyme | the cleavage site of the enzyme) |
| Bsu Endo V | 5′-CAGGGATCCGTGAAGCTATCCUG ↑ CGT |
| | CTAGGACTAAGC-3′ |
| C3 | 5′-CAGGGATCCGTGAAGCTATCC ↑ UGCGT |
| | CTAGGACTAAGC-3′ |

### Example 8: Deoxyinosine and Deoxyuridine Recognition and DNA-Strand Cleavage by Endo V and Endo Q at Three Different Reaction Temperatures

The sample groups include (1) only the DNA substrate(S) that serves as a negative control; (2) an in-house E. coli endonuclease V (Eco EndoV), which can be referred to US 2021/0254114A1; (3) a Pyrococcus furiosus endonuclease V (Pfu Endo V): (4) a Pyrococcus furiosus endonuclease Q (Pfu Endo Q): (5) a Thermococcus barophilus endonuclease V (Tba Endo V); and (6) a Bacillus pumilus endonuclease Q (Bpu Endo Q) (SEQ ID NO: 12). The sample mixture (10 ul) containing a 100 nM of a single-stranded Hex-Top-U38mer DNA substrate or Hex-Top-138mer DNA substrate were incubated with 200 nM endonuclease of each sample group in the enzyme reaction buffer. The sample mixtures were incubated at 37° C., 55° C., or 60° C., respectively, for 20 minutes. Each enzyme reaction mixture was stopped by the addition of equal volume (10 μL) of 2× quench solution.

The total 20 μL of sample were denatured at 95° C. for 10 min, and 4 μL of each sample mixture were analyzed by 20% denaturing polyacrylamide gel electrophoresis containing 8 M urea in the 1× TBE buffer (90 mM Tris-base, 90 mM boric acid, and 2 mM EDTA). The results of the gel were then visualized by Amersham Typhoon scanner (Cytiva, Marlborough, MA).

The results showed that both Pfu Endo V and Q site-specifically cleave both the free form and immobilized DNA strand containing dexovinosine or deoxyuridine at 55° C. and 60° C.: Tba Endo V site-specifically cleaves both the free form and immobilized DNA strand containing deoxyuridine at 55° C.; and Bpu EndoQ site-specifically cleave both the free form and immobilized DNA strand containing dexovinosine or deoxyuridine at 37° C. On the contrary, E. coli Endo V only cleaves the DNA strand containing a deoxyinosine, but not a deoxyuridine, at 37° C.

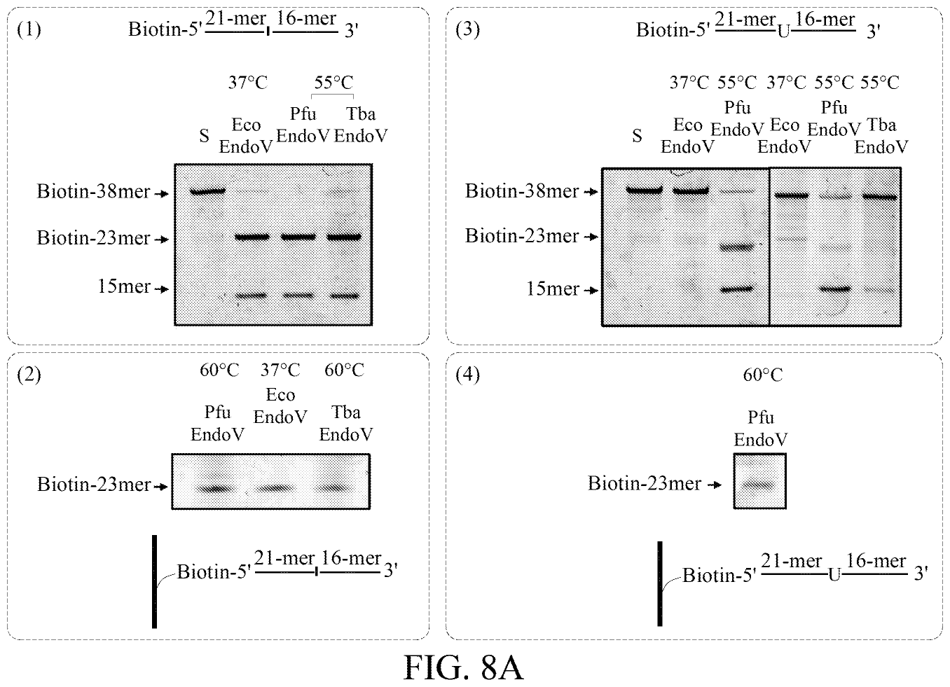

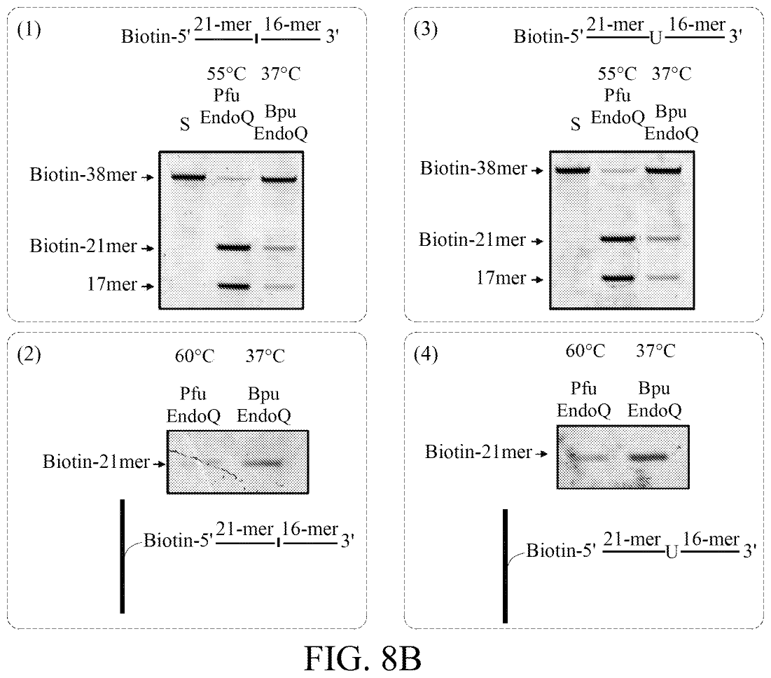

As shown in FIGS. 8A, 8B, and Tables 10 and 11 below, Pfu Endo V and Tba Endo V efficiently recognize deoxyinosine (I) or deoxyuridine (U) and cleaves the phosphodiester bond between the first nucleotide (G) and the second nucleotide (C) starting from the deoxyinosine (I) or deoxyuridine (U) toward 3′ end of the DNA, thereby releasing a 15-mer single, or double-stranded, DNA and the remaining 23-mer single, or double-stranded, DNA with a free 3′-hydroxyl group at the 3′-terminus, which can readily serve as a new, or reusable, initiator for the next-round of nucleic acid synthesis. By contrast, Eco Endo V can only perform such activity on the DNA containing a deoxyinosine. In addition, both Pfu Endo Q and Bpu Endo Q efficiently recognize deoxyinosine (I) or deoxyuridine (U) and cleave the phosphodiester bond between the deoxyinosine (I), or deoxyuridine (U), and the first nucleotide (C) starting from the deoxyinosine (I), or deoxyuridine (U), toward the 5′ end of the DNA at the reaction temperature, thereby releasing a 17-mer single, or double-stranded, DNA and the remaining 21-mer single, or double-stranded, DNA with a free 3′-hydroxyl group at the 3′-terminus, which can readily serve as a new, or reusable, initiator for the next-round of nucleic acid synthesis.

Also, both Pfu Endo V and Q site-specifically and efficiently cleaves the DNA strand containing either a deoxyinosine or a deoxyuridine at both 55° C. and 60° C.: Tba Endo V site-specifically and efficiently cleaves the DNA strand containing either a deoxyinosine or a deoxyuridine at both 55° C. and 60° C. y; and Bpu Endo Q site-specifically and efficiently cleaves the DNA strand containing either a deoxyinosine or a deoxyuridine at 37° C. By contrast, the E. coli Endo V only cleaves the DNA strand containing a deoxyuridine at 37° C.

Given the foregoing, Pfu Endo V, Pfu Endo Q, Tba Endo V, and Bpu Endo Q can site-specifically recognize a guiding nucleotide, efficiently cleave the nucleic acid strand according to the position of the guiding nucleotide, and effectively regenerate an initiator with a free 3′-hydroxyl group at the 3′-terminusat a wide range of reaction temperatures including ambient reaction temperatures.

TABLE 10
Site-specific recognition and cleavage
of Hex-Top-I38-mer and Hex-Top-
U38-mer by Eco, Pfu, and Tba Endo V

| | Site-specific cleavage of |
|---|---|
| | Hex-Top-I38-mer and Hex-Top-U38-mer |
| | DNA (SEQ ID NO: 5) (↑ refers to |
| Enzyme | the cleavage site of the enzyme) |
| Eco Endo V, | Hex-Top-I38-mer: |
| Pfu Endo V, | 5′-CAGGGATCCGTGAAGCTATCCIG ↑ CGTCTA |
| Tba Endo V | GGACTAAGC-3′ |
| | Hex-Top-U38-mer: |
| | 5′-CAGGGATCCGTGAAGCTATCCUG ↑ CGTCTA |
| | GGACTAAGC-3′ |

TABLE 11
Site-specific recognition and cleavage
of Hex-Top-I38-mer and Hex-Top-
U38-mer by Pfu and Bpu EndoQ

| | Site-specific cleavage of |
|---|---|
| | Hex-Top-I38-mer and Hex-Top-U38-mer |
| | DNA (SEQ ID NO: 5) (↑ refers to |
| Enzyme | the cleavage site of the enzyme) |
| Pfu Endo Q, | Hex-Top-I38-mer: |
| Bpu Endo Q | 5′-CAGGGATCCGTGAAGCTATCC ↑ IGCGTCTA |
| | GGACTAAGC-3′ |
| | Hex-Top-U38-mer: |
| | 5′-CAGGGATCCGTGAAGCTATCC ↑ UGCGTCTA |
| | GGACTAAGC-3′ |

The present disclosure describes with embodiments thereof, and it is understood that various modifications, without departing from the scope of the present disclosure, are in accordance with the embodiments of the present disclosure. Hence, the embodiments described are intended to cover the modifications within the scope of the present disclosure, rather than to limit the present disclosure. The scope of the claims therefore should be accorded the broadest interpretation so as to encompass all such modifications.

What is claimed is:

1. A method of enzymatically synthesizing a polynucleotide, comprising:
providing an initiator comprising a 3′-terminal nucleotide having a free 3′-hydroxyl group:
incorporating nucleotide monomers to the initiator by a polymerase to elongate a nucleic acid strand from the free 3′-hydroxyl group, wherein the nucleotide monomers comprise a guiding nucleotide to be recognized by an endonuclease, such that the guiding nucleotide is incorporated at a specific position of the nucleic acid strand; and
subjecting the endonuclease to cleave the nucleic acid strand according to the position of the guiding nucleotide to release a predetermined polynucleotide and leaves a remaining nucleic acid strand having a new free 3′-hydroxy group, wherein the endonuclease specifically recognizes the guiding nucleotide and cleaves at a second phosphodiester bond 3′ to the guiding nucleotide, a first phosphodiester bond 5′ to the guiding nucleotide, a second phosphodiester bond 5′ to the guiding nucleotide, or a third phosphodiester bond 5′ to the guiding nucleotide, so that the predetermined polynucleotide is obtained and the remaining nucleic acid strand having the new free 3′-hydroxyl group is retained and serves as a new initiator for another round of polynucleotide synthesis.

2. The method of claim 1, the nucleic acid strand is synthesized in a template-independent manner.

3. The method of claim 1, the nucleic acid strand is synthesized in a template-directed manner.

4. The method of claim 1, wherein a nucleobase of the guiding nucleotide is uracil, xanthine, or hypoxanthine.

5. The method of claim 1, wherein the guiding nucleotide contains an uracil or inosine.

6. The method of claim 1, wherein the polymerase is a B-family DNA polymerase.

7. The method of claim 1, wherein the endonuclease derives from Thermococcus barophilus (Tba), Pyrococcus furiosus (Pfu), Methanosarcina acetivorans (Mac). Bacillus pumilus (Bpu), Pyrococcus abyssi (Pab), Thermococcus kodakarensis (Tko), Thermococcus gammatolerans (Tga), or Bacillus subtilis (Bsu).

8. The method of claim 7, wherein the endonuclease is selected from the group consisting of Thermococcus barophilus endonuclease V (Tba Endo V), Bacillus subtilis endonuclease V (Bsu Endo V), Pyrococcus furiosus endonuclease V (Pfu Endo V), Thermococcus kodakarensis endonuclease V (Tko Endo V), Pyrococcus furiosus endonuclease Q (Pfu Endo Q), Methanosarcina acetivorans endonuclease Q (Mac Endo Q), Bacillus pumilus endonuclease Q (Bpu Endo Q), Pyrococcus abyssi NucS endonuclease (Pab NucS), Thermococcus kodakarensis EndoMS endonuclease (Tko EndoMS: SEQ ID NO: 17), Thermococcus gammatolerans NucS endonuclease (Tga NucS), and the variants thereof.

9. The method of claim 1, wherein the endonuclease is selected from the group consisting of Bacillus subtilis endonuclease V (Bsu Endo V), Escherichia coli endonuclease V (Eco Endo V), Pyrococcus furiosus endonuclease V (Pfu Endo V), Thermococcus barophilus endonuclease V (Tba Endo V), Thermococcus kodakarensis endonuclease V (Tko Endo V) and the enzyme variants thereof to cleave at the second phosphodiester bond 3′ to the guiding nucleotide.

10. The method of claim 1, wherein the endonuclease is selected from the group consisting of Pyrococcus furiosus endonuclease Q (Pfu Endo Q), Methanosarcina acetivorans endonuclease Q (Mac Endo Q), Bacillus pumilus endonuclease Q (Bpu Endo Q) and the enzyme variants thereof to cleave at the first phosphodiester bond 5′ to the guiding nucleotide.

11. The method of claim 1, wherein the endonuclease is selected from the group consisting of Thermococcus gammatolerans NucS endonuclease (Tga NucS), Pyrococcus abyssi NucS endonuclease (Pab NucS) and the enzyme variants thereof to cleave at the second phosphodiester bond 5′ to the guiding nucleotide.

12. The method of claim 1, wherein the endonuclease is selected from the group consisting of Thermococcus kodakarensis EndoMS endonuclease (Tko EndoMS) and the enzyme variants thereof to cleave at the third phosphodiester bond 5′ to the guiding nucleotide.

13. The method of claim 1, which is performed at a temperature ranging from 10° C. to 100° C.

14. The method of claim 1, wherein the initiator is immobilized to a solid support with the 5′ end.

15. A method of enzymatically retrieving a polynucleotide, comprising:
providing a synthetic polynucleotide having a guiding nucleotide to be recognized by an endonuclease specifically; and
subjecting the endonuclease to cleave the synthetic polynucleotide according to a position of the guiding nucleotide to release a predetermined polynucleotide, wherein the endonuclease recognizes the guiding nucleotide and cleaves at a second phosphodiester bond 3′ to the guiding nucleotide, a first phosphodiester bond 5′ to the guiding nucleotide, a second phosphodiester bond 5′ to the guiding nucleotide, or a third phosphodiester bond 5′ to the guiding nucleotide, so that the predetermined polynucleotide is obtained.

16. A kit for enzymatically synthesizing a polynucleotide, comprising:
an initiator comprising a 3′-terminal nucleotide having a free 3′-hydroxyl group:
a polymerase for incorporating nucleotide monomers to the initiator to elongate a nucleic acid strand from the free 3′-hydroxyl group, wherein the nucleotide monomers comprise a guiding nucleotide, such that the guiding nucleotide is incorporated in the nucleic acid strand; and
an endonuclease for cleaving the nucleic acid strand according to a position of the guiding nucleotide to release a predetermined polynucleotide and leave a new free 3′-hydroxyl group at the 3′ end of a remaining nucleic acid strand, wherein the endonuclease specifically recognizes the guiding nucleotide and cleaves at a second phosphodiester bond 3′ to the guiding nucleotide, a first phosphodiester bond 5′ to the guiding nucleotide, a second phosphodiester bond 5′ to the guiding nucleotide, or a third phosphodiester bond 5′ to the guiding nucleotide, so that the predetermined polynucleotide is obtained and the remaining nucleic acid strand having the new free 3′-hydroxyl group is retained and serves as a new initiator for another round of polynucleotide synthesis.

17. A kit of enzymatically retrieving a polynucleotide, the kit comprising:
a synthetic polynucleotide having a guiding nucleotide; and
an endonuclease for cleaving the synthetic polynucleotide to release a predetermined polynucleotide, wherein the endonuclease recognizes the position of the guiding nucleotide and cleaves at a second phosphodiester bond 3′ to the guiding nucleotide, a first phosphodiester bond 5′ to the guiding nucleotide, a second phosphodiester bond 5′ to the guiding nucleotide, or a third phosphodiester bond 5′ to the guiding nucleotide, so that the predetermined polynucleotide is obtained.

18. The kit of claim 16 or 17, wherein the guiding nucleotide contains uracil or inosine.


SEQUENCE LISTING (17 sequences)

SEQ ID NO: 1 - DNA, 45 bp, synthetic construct
  source 1..45: mol_type = other DNA
ctcggcctggcacaggtccgttcagtgctgcggcgaccaccgagg

SEQ ID NO: 2 - DNA, 46 bp, synthetic construct
  source 1..46: mol_type = other DNA
  modified_base 46: mod_base = OTHER; note = uracil
ctcggcctggcacaggtccgttcagtgctgcggcgaccaccgaggt

SEQ ID NO: 3 - DNA, 47 bp, synthetic construct
  source 1..47: mol_type = other DNA
  modified_base 46: mod_base = OTHER; note = uracil
  modified_base 47: mod_base = OTHER; note = 3&apos;-O-azidomethyl-2&apos;-deoxyguanosine
ctcggcctggcacaggtccgttcagtgctgcggcgaccaccgaggtn

SEQ ID NO: 4 - DNA, 38 bp, synthetic construct
  source 1..38: mol_type = other DNA
  modified_base 22: mod_base = OTHER; note = uracil
cagggatccgtgaagctatcctgcgtctaggactaagc

SEQ ID NO: 5 - DNA, 38 bp, synthetic construct
  source 1..38: mol_type = other DNA
  modified_base 22: mod_base = i
cagggatccgtgaagctatccngcgtctaggactaagc

SEQ ID NO: 6 - DNA, 38 bp, synthetic construct
  source 1..38: mol_type = other DNA
gcttagtcctagacgcaggatagcttcacggatccctg

SEQ ID NO: 7 - protein, 223 aa, Escherichia coli
MDLASLRAQQIELASSVIREDRLDKDPPDLIAGADVGFEQGGEVTRAAMVLLKYPSLELVEYKVARIATTMPYIPGFLSFREYPALLAAWEMLSQKPDLVFVDGHGISHPRRLGVASHFGLLVDVPTIGVAKKRLCGKFEPLSSEPGALAPLMDKGEQLAWVWRSKARCNPLFIATGHRVSVDSALAWVQRCMKGYRLPEPTRWADAVASERPAFVRYTANQP

SEQ ID NO: 8 - protein, 190 aa, Pyrococcus furiosus
IDLRKLTEVQRRLSRKIVEKPIDIAKVKRVGAVDVSYKNNFAKAAFVCVEFPSGEIIKTKTIVTTVEFPYIPTFFFLRETKPILLAVKDENFDVLLVEGHGKAHPRRYGLASHIGVILSKPTIGVAKRLLRGVSKDTYVKVGKAFVSVGNLITLNDAVRIVEKLLDENGYPKPLNIADKLSKRGESREKE

SEQ ID NO: 9 - protein, 424 aa, Pyrococcus furiosus
MIVDGDLHIHSHYSKAVSKLMTFPIIAENAKLKGLNLVGTGDSLNPHWEKELLKHSKPIDDGTFEVNGVKFILTCEVEDKRRVHHLLIFPTLSQVREFREKVKIYSTNIESEGRPNLNLTAEEIAEMANELDILIGPAHAFTPWTSLYKEYDSLKDAYGDAKIDFLELGLSADSDMADMIKAHHSIPYLSNSDAHSPNPHRLGREFNRFEVKDVTFEEIRKAIKGVGGRKIMLNAGLDPRLGKYHLTACSRCYTKYTLQDAVSLSWKCPKCGGIIKKGVRDRILELADTSEKPKDRPPYVRLAPLAEIIAMVLGKGIESKAVKLLWNRFLREFGSEIRVLIDLPIESIASVHEGVAKAIWAYRNNKLIIVPGGGGKYGEIRIPEEILKAKIEDLNSIEISGGEAELPKPKQRTLLQYIKNKEVN

SEQ ID NO: 10 - protein, 189 aa, Thermococcus barophilus
SRANVNFKKIAEVQRKLSKRIVEKPLDISRIKTIGAVDVSYKKGKAKAAFVLCSFPSCEILKAKVIETNVDFPYIPTFFFLRETRPVLLAIRGETFDVLLVEGHGKAHPRGYGLASHIGLLLKKPTIGIAKKPLRGYPEGSLVKVGKAYVSVGNLIDLNSAAKIVEMVNENGYPKPLRIADKLSKGAEK

SEQ ID NO: 11 - protein, 237 aa, Bacillus subtilis
KVFDVHKFDMKKEQDFLQVQFNLKNRINLSPTIHPDSINTCAGVDLAYWEQDGEPYGVCCIIVIDADTKEVIEKVHSMGRISVPYVSGFLAFRELPLIIEAAKKLETEPDVFLFDGNGYLHYNHMGVATHAAFFLGKPTIGIAKTYLKIKGCDFVTPEIEVGAYTDIIIDGEVYGRALRTRRDVKPIFLSCGNYIDLDSSYQITMSLINQESRLPIPVRLADLETHVLRTFYQKNHV

SEQ ID NO: 12 - protein, 393 aa, Bacillus pumilus
MKEFFADIHIHIGRTRTGRAVKITGARSLTIDQILIEAAESKGMGMIGVIDAQSPEVLEELIEGVEEGRYTELNDGGLSFGQTVLLLGSELEINDDHSKGPIHVLAFMPTLHKMSEFSAWLSLHMKNVHLSSQRLYVDGKILQRKVKELGGLFIPAHIFTPHKSLYGKGVNTSLTEVFDPDLIDAVELGLSCDTSMASQLSELNRYPFLTNSDAHSLGKIAREYTKIKMDHASFAEFKLALEEKDGRAVTGNYGLAPRLGKYYHTTCEKCGVRPHAPDQEKCHVCGHTRMTKGVSERLKELADQESEKGTRPPYVHQLPLQFIPGVGPKTLEKLKAVFHTEMNILHQATEQELKAVLPEKTAGYIIKARKGEVELIAGGGGVYGKVDINHESH

SEQ ID NO: 13 - protein, 251 aa, Pyrococcus abyssi
MRKVIIKENPSEEEIKELLDLAEKHGGVVTIFARCKVHYEGRAKSELGEGDRIIIIKPDGSFLIHQNKKREPVNWQPPGSKVTFKENSIISIRRRPYERLEVEIIEPYSLVVFLAEDYEELALTGSEAEMANLIFENPRVIEEGFKPIYREKPIRHGIVDVMGVDKDGNIVVLELKRRKADLHAVSQLKRYVDSLKEEYGENVRGILVAPSLTEGAKKLLEKEGLEFRKLEPPKKGNEKRSKQKTLDFFTP

SEQ ID NO: 14 - protein, 467 aa, Methanosarcina acetivorans
MKVNADLHLHSKYSMACSGKMELPTIASEASKKGMELIGTGDCTHPEWLKDIKKAAISDEEIRIGNTFFIPTTEIEDSNRVHHLLILSSVSKAEELAERIAPFGNLEADGRPTVKLDGCEIAEIAKDLEALIGPCHAFTPWTALYAYHDSLESCYGDMIDYISFIELGLSADSDYADRIEELQRLTFLTNSDAHSPYTNKLAREFTQFNVPDITFEGLRKAILREQGYGPTLNVGFFPEEGKYNRSACIKCFTRYTLPEAEANRWRCPKCGGIMKKGVADRVNELADFETPRHPDHRPPYLHLIPLAEIIQMALGHASIQTKGVKTAWETLVENFGNEVEALIYAEPDALGIVDDRIVNAILAFRKGNVIIHPGGGGQYGWLELPKPLKAEKPGTAEKSAGEKKKTSKTTKREKEKTGAQISFADLESIKSAELAKTTEENTAGEEAKTPEEKGPEKPAGQKSLFDF

SEQ ID NO: 15 - protein, 249 aa, Thermococcus gammatolerans
MPKVELRENPSPEEIKLLVDLAISSEGVLTIFARCRVHYDGRAKSELGPGDRVIIVKPDGSFLIHQKEKREPVNWQPPGSVVRLELREKPVLISVRRKPRETLEVELDEVYLITVFHAEDYEELALTGSEAEMAELIFENPEVIELGFKPLYREKPIRHGIVDVLGVDRDGNLVVLELKRRRADLHAVSQLKRYVETLREEHENVRGILVAPSLTSGAKKLLEKEGLEFRKLEPPKRDGKSRGRQLRLF

SEQ ID NO: 16 - protein, 197 aa, Thermococcus kodakarensis
MSEGLFKKLEEVQRKLAERIVERPLEVSKIKTVGAVDVSYRDERARAAFVLCSFPDCELLKQRVVEVDVSFPYIPTFFFLRETRPVLIALGKERPDVLLVEGHGRAHPRGYGLASHIGLVLGIPTIGISKRLLRGTPEGSWVKVGKAYVSVGHLIDLPSAVEVVKTLNKNGYPLPLRIADRLSRGHTSWSGGVNDEH

SEQ ID NO: 17 - protein, 252 aa, Thermococcus kodakarensis
MSKDKVTVITSPSTEELVSLVNSALLEEAMLTIFARCKVHYDGRAKSELGSGDRVIIVKPDGSFLIHQSKKREPVNWQPPGSRVRLELRENPVLVSIRRKPRETLEVELEEVYMVSVFRAEDYEELALTGSEAEMAELIFENPEVIEPGFKPLFREKAIGTGIVDVLGRDSDGNIVVLELKRRRAELHAVRQLKSYVEILREEYGDKVRGILVAPSLTSGAKRLLEKEGLEFRKLEPPKRDSKKKGRQKTLF